(19) United States
(12) Patent Application Publication
(10) Pub. No.: US 2026/0168904 A1
(43) Pub. Date: Jun. 18, 2026

(54) SYSTEMS, METHODS, AND APPARATUS FOR CONCRETE QUALITY INSPECTION

(71) Applicant: Gecko Robotics, Inc., Pittsburgh, PA (US)

(72) Inventors: Alexander R. Cuti, Pittsburgh, PA (US); Dillon R. Jourde, Pittsburgh, PA (US); Ryan List, Beaver, PA (US); Michael A. Binger, Pittsburgh, PA (US); Samuel Theodore Westenberg, Portland, ME (US); Edwin H. Cho, Pittsburgh, PA (US); Edward A. Bryner, Pittsburgh, PA (US); Troy Demmer, Glenshaw, PA (US); Lincoln Roop, Pittsburgh, PA (US); Larry Duane Olson, Golden, CO (US); Dennis Alan Sack, Boulder, CO (US); Weronika van Vianen, Philadelphia, PA (US)

(21) Appl. No.: 19/418,131

(22) Filed: Dec. 12, 2025

Related U.S. Application Data
(63) Continuation of application No. PCT/US24/33851, filed on Jun. 13, 2024.
(60) Provisional application No. 63/507,945, filed on Jun. 13, 2023.

(51) Int. Cl. G01N 3/30; G01N 33/38

(52) U.S. Cl. CPC G01N 3/30; G01N 33/383

(57) ABSTRACT

A payload for an inspection robot to inspect an inspection surface may include a concrete sensing assembly and a payload mount. The concrete sensing assembly may include a first impactor at a first end, a first near transducer at a first distance from the first impactor, and a second far transducer at a second distance from the first impactor. The second distance may be greater than the first distance. The payload mount may be structured to couple the concrete sensing assembly to the inspection robot and to move the concrete sensing assembly between at least a first lowered position and a second raised position.

### CROSS-REFERENCE TO RELATED APPLICATIONS

This application claims benefit of and is a continuation of International Patent Application No. PCT/US2024/033851 (Attorney Docket No. GROB-0026-WO), filed Jun. 13, 2024, and entitled “SYSTEMS, METHODS, AND APPARATUS FOR CONCRETE QUALITY INSPECTION,” International Pub. No. WO 2024/259133, which is hereby incorporated by reference in its entirety for all purposes.

International Patent Application No. PCT/US2024/033851 claims the benefit of U.S. Provisional Patent Application 63/507,945, filed Jun. 13, 2023, and entitled “INSPECTION ROBOT FOR CONCRETE QUALITY INSPECTION” (Attorney Docket No. GROB-0026-P01).

Each of the foregoing applications is incorporated herein by reference in the entirety for all purposes.

### BACKGROUND

Previously known systems suffer from a number of challenges, for example ensuring that sensors of an inspection robot payload are able to obtain effective readings from an inspection surface.

### SUMMARY

In some aspects, the techniques described herein relate to an inspection robot, including: a means for motive operation of an inspection robot body on an inspection surface; a payload including an impactor positioned at a first end of the payload, a first near transducer positioned at a selected first distance from the impactor, and a second far transducer positioned at a selected second distance from the impactor, where the selected second distance is greater than the selected first distance; and a means for data and command communication between the payload and a computing device positioned on the inspection robot body.

In some aspects, the techniques described herein relate to an inspection robot, further including a second impactor positioned at a second opposite end of the payload, at the selected first distance from the second far transducer.

In some aspects, the techniques described herein relate to an inspection robot, wherein the inspection robot is positioned on a metal surface side of the inspection surface, wherein the inspection surface further includes a concrete layer positioned on an opposing side of the metal surface side, and wherein the computing device includes an analysis component that determines a presence of a defect in the concrete layer in response to a compression wave analysis determined in response to data from the impactor and the first near transducer.

In some aspects, the techniques described herein relate to an inspection robot, wherein the analysis component further determines a characteristic of the defect in response to a shear wave analysis determined in response to data from the impactor, the first near transducer, and the second far transducer.

In some aspects, the techniques described herein relate to an inspection robot, wherein the analysis component further determines a bonding characteristic of the concrete layer in response to at least one of the compression wave analysis or the shear wave analysis.

In some aspects, the techniques described herein relate to an inspection robot, wherein the first near transducer further includes a selected one of a group of transducers mounted on a wheel, the group of transducers including a number of transducers sequentially coupling to the inspection surface in response to a rotation of the wheel.

In some aspects, the techniques described herein relate to an inspection robot, wherein the first payload further includes an encoder wheel configured to contact the inspection surface, wherein the analysis component determines the selected one of the group of transducers in response to data from the encoder wheel.

In some aspects, the techniques described herein relate to an inspection robot, wherein the inspection surface includes a concrete surface.

In some aspects, the techniques described herein relate to an inspection robot, wherein the defect in the concrete layer includes at least one defect selected from: a void, a crack, a debonded portion, a delaminated portion, a honeycombed portion, corrosion, a cold joint, soil chemical corrosion, or rebar spacing.

In some aspects, the techniques described herein relate to an inspection robot, further including: wherein the impactor, first transducer, and second transducer include a first concrete sensing assembly; and wherein the payload further includes at least one additional concrete sensing assembly, each horizontally distributed on the payload.

In some aspects, the techniques described herein relate to a payload for an inspection robot to inspect an inspection surface, the payload including: a concrete sensing assembly including a first impactor at a first end, a first near transducer at a first distance from the first impactor, and a second far transducer at a second distance from the first impactor, wherein the second distance is greater than the first distance; and a payload mount structured to: couple the concrete sensing assembly to the inspection robot; and move the concrete sensing assembly between at least a first lowered position and a second raised position.

In some aspects, the techniques described herein relate to a payload, further including: the concrete sensing assembly further including a second impactor positioned at a second end, wherein the second end is opposite to the first end, the second far transducer is at the first distance from the second impactor, and the first near transducer is at the second distance from the second impactor.

In some aspects, the techniques described herein relate to a payload, wherein: when the concrete sensing assembly is in the first lowered position, the first impactor is in contact with the inspection surface; and when the concrete sensing assembly is in the second raised position, the first impactor is spaced apart from the inspection surface.

In some aspects, the techniques described herein relate to a payload, wherein the payload mount includes a downforce structure to provide a selected downforce of the concrete sensing assembly including the first impactor against the inspection surface.

In some aspects, the techniques described herein relate to a payload, wherein the downforce structure includes at least one of an actuator or a spring.

In some aspects, the techniques described herein relate to a payload, wherein the payload mount includes a spring-based structure having a plurality of springs to provide a selected downforce of the concrete sensing assembly against the inspection surface when the concrete sensing assembly is in the first lowered position and to provide a selected upforce of the concrete sensing assembly away from the inspection surface when the concrete sensing assembly is in the second raised position.

In some aspects, the techniques described herein relate to a payload, wherein the payload mount includes at least one actuator structured to move the concrete sensing assembly between the first lowered position and the second raised position.

In some aspects, the techniques described herein relate to a payload, wherein the first impactor includes a piezoelectric material to provide an impact against the inspection surface when the piezoelectric material is provided with an electric charge.

In some aspects, the techniques described herein relate to a system, including: the payload; and the inspection robot.

In some aspects, the techniques described herein relate to a system, wherein the inspection robot includes a controller to instruct the payload mount to move the concrete sensing assembly between the first lowered position and the second raised position.

In some aspects, the techniques described herein relate to a system, wherein the controller instructs the payload mount to move the concrete sensing assembly to the second raised position when the inspection robot detects an obstacle on the inspection surface in a direction of travel of the inspection robot.

In some aspects, the techniques described herein relate to a system, further including: a first near transducer wheel including the first near transducer; a second far transducer wheel including the second far transducer; and another concrete sensing assembly including a third transducer wheel having a third transducer, wherein the controller instructs the payload mount to move the concrete sensing assembly between the first lowered position and the second raised position to change a sensing phase of at least one of the first near transducer or the second far transducer relative to the third transducer.

In some aspects, the techniques described herein relate to a payload for an inspection robot to inspect an inspection surface, the payload including: a concrete sensing assembly including at least one transducer and at least one impactor, the at least one transducer at a first distance from the at least one impactor on a horizontal axis to define a first inspection depth and a first horizontal inspection width, wherein the first horizontal inspection width corresponds to the first distance; and a payload mount including a rastering actuator, the payload mount structured to couple the concrete sensing assembly to the inspection robot, wherein the rastering actuator is structured to move the concrete sensing assembly between a first horizontal position and a second horizontal position on the horizontal axis to provide a rastered horizontal inspection width that is greater than the first horizontal inspection width.

In some aspects, the techniques described herein relate to a payload, wherein the horizontal axis is orthogonal to a direction of travel of the inspection robot.

In some aspects, the techniques described herein relate to a payload, wherein the at least one transducer includes a plurality of transducers and the at least one impactor includes a plurality of impactors, and at least some of the plurality of impactors correspond to respective ones of the plurality of transducers.

In some aspects, the techniques described herein relate to a payload, wherein: the plurality of transducers and the plurality of impactors define a plurality of horizontal inspection widths corresponding to respective distances between the plurality of transducers and the plurality of impactors; and the rastering actuator is structured to move the concrete sensing assembly between the first horizontal position and the second horizontal position to provide a plurality of rastered horizontal inspection widths that are greater than the respective plurality of horizontal inspection widths.

In some aspects, the techniques described herein relate to a system, including: the payload; and the inspection robot.

In some aspects, the techniques described herein relate to a system, wherein the inspection robot includes a controller to instruct the rastering actuator to move the concrete sensing assembly in two opposite directions between the first horizontal position and the second horizontal position.

In some aspects, the techniques described herein relate to a system, wherein the controller instructs the rastering actuator to move the concrete sensing assembly in at least one of the two opposite directions when the inspection robot is not moving in the inspection direction.

In some aspects, the techniques described herein relate to a payload for an inspection robot to inspect an inspection surface, the payload including: a plurality of concrete sensing assemblies including a first plurality of concrete sensing assemblies horizontally distributed across the payload relative to a direction of travel of the inspection robot, wherein at least one of the first plurality of concrete sensing assemblies includes an impactor and a transducer; and a payload mount structured to couple the concrete sensing assemblies to the inspection robot.

In some aspects, the techniques described herein relate to a payload, wherein at least some of the plurality of concrete sensing assemblies include at least one transducer and at least one impactor.

In some aspects, the techniques described herein relate to a payload, wherein the plurality of concrete sensing assemblies further includes at least one concrete sensing assembly positioned forward of the first plurality of concrete sensing assemblies relative to the direction of travel of the inspection robot to provide increased horizontal resolution of an inspection operation of the inspection surface.

In some aspects, the techniques described herein relate to a payload, wherein the at least one concrete sensing assembly is positioned at a gap position to cover a horizontal gap between two or more of the first plurality of concrete sensing assemblies.

In some aspects, the techniques described herein relate to a payload, wherein the at least one concrete sensing assembly is positioned at or near a horizontal center of the payload to inspect a feature of the inspection surface.

In some aspects, the techniques described herein relate to a payload, wherein the feature includes a weld line.

In some aspects, the techniques described herein relate to a payload, wherein the at least one concrete sensing assembly includes a sensing package that is distinct from a sensing package of at least one of the first plurality of concrete sensing assemblies.

In some aspects, the techniques described herein relate to a payload, wherein the plurality of concrete sensing assemblies further includes a plurality of forward concrete sensing assemblies positioned forward of the first plurality of concrete assemblies relative to the direction of travel of the inspection robot, and wherein the plurality of forward concrete sensing assemblies are positioned at respective gap positions to cover horizontal gaps between respective two or more of the first plurality of concrete sensing assemblies.

In some aspects, the techniques described herein relate to a system, including: the payload; and the inspection robot.

In some aspects, the techniques described herein relate to a payload for an inspection robot to inspect an inspection surface, the payload including: a concrete sensing assembly including: at least a first transducer wheel; and at least a first impactor structured to provide an acoustic impact against the inspection surface, the first transducer wheel including a first wheel and at least one transducer to sense acoustic waves produced by the at least one impactor providing the acoustic impact against the inspection surface.

In some aspects, the techniques described herein relate to a payload, the first transducer wheel including: the at least one transducer within the first wheel, wherein the first wheel acoustically couples the at least one transducer to the inspection surface.

In some aspects, the techniques described herein relate to a payload, wherein the at least one transducer includes a single transducer.

In some aspects, the techniques described herein relate to a payload, wherein the single transducer is within a bearing of the first transducer wheel and remains in a fixed orientation relative to the inspection surface while the first wheel rotates on the inspection surface.

In some aspects, the techniques described herein relate to a payload, wherein the at least one transducer includes a plurality of transducers facing radially outward from the first wheel.

In some aspects, the techniques described herein relate to a payload, wherein the plurality of transducers are on an exterior of the first wheel and are spaced equally around a circumference of the first wheel.

In some aspects, the techniques described herein relate to a payload, wherein the plurality of transducers are within a material of the first wheel.

In some aspects, the techniques described herein relate to a payload, wherein the plurality of transducers are spaced around a circumference of the first wheel such that each of the plurality of transducers sequentially contacts the inspection surface as the first wheel rolls over the inspection surface.

In some aspects, the techniques described herein relate to a payload, wherein a number of the plurality of transducers is based on a desired inspection resolution.

In some aspects, the techniques described herein relate to a payload, wherein the number of the plurality of transducers is between three and twelve, inclusive.

In some aspects, the techniques described herein relate to a payload, wherein: the concrete sensing assembly includes a plurality of transducer wheels including the first transducer wheel and a second transducer wheel; and the first transducer wheel includes a first plurality of transducers including the at least one transducer, and the second transducer wheel includes a second plurality of transducers.

In some aspects, the techniques described herein relate to a payload, wherein: the concrete sensing assembly is structured such that as the inspection robot moves in a direction of travel along the inspection surface, the first transducer wheel and the second transducer wheel both turn and transducers of the first plurality of transducers and transducers of the second plurality of transducers alternate in sequentially contacting the inspection surface such that inspection data provided by the first plurality of transducers is out of phase with inspection data provided by the second plurality of transducers.

In some aspects, the techniques described herein relate to a payload, wherein the acoustic waves include a compression wave and a shear wave.

In some aspects, the techniques described herein relate to a payload for an inspection robot to inspect an inspection surface, including: a concrete sensing assembly including: at least a first transducer wheel; and at least a first impactor to provide an acoustic impact against the inspection surface, the first transducer wheel including a first wheel and at least one transducer within the first wheel to sense acoustic waves produced by the at least one impactor providing the acoustic impact against the inspection surface.

In some aspects, the techniques described herein relate to a payload, wherein the at least one transducer includes a plurality of transducers spaced apart from each other within the first wheel.

In some aspects, the techniques described herein relate to a payload, wherein the at least one transducer is radially inward of the inspection surface when the first wheel contacts the inspection surface.

In some aspects, the techniques described herein relate to a payload, wherein the at least one transducer is within a radial through-hole of the first wheel.

In some aspects, the techniques described herein relate to a payload, wherein the radial through-hole provides a couplant chamber to be filled with couplant to acoustically couple the at least one transducer to the inspection surface.

In some aspects, the techniques described herein relate to a payload, wherein a material of the first wheel is between the at least one transducer and the inspection surface and acoustically couples the at least one transducer to the inspection surface.

In some aspects, the techniques described herein relate to a payload, wherein the material is selected to correspond to an acoustic property of the inspection surface.

In some aspects, the techniques described herein relate to a payload for an inspection robot to inspect an inspection surface, including: a concrete sensing assembly including a plurality of impactors and a plurality of transducer wheels distributed at respective horizontal positions along a horizontal axis, wherein an orientation of the horizontal axis is different from a direction of travel of the inspection robot, and wherein there are a greater number of the plurality of transducer wheels than a number of the plurality of impactors; and the plurality of transducer wheels and the plurality of impactors distributed at the respective horizontal positions to provide for wave analysis at selected inspection depths, the selected inspection depths determined by distances between the plurality of transducer wheels and the plurality of impactors along the horizontal axis.

In some aspects, the techniques described herein relate to a payload, wherein outermost transducer wheels of the plurality of transducer wheels at respective first and second ends of the concrete sensing assembly each correspond to respective outermost impactors of the plurality of impactors at the first and second ends of the concrete sensing assembly.

In some aspects, the techniques described herein relate to a payload, wherein the plurality of impactors includes only the outermost impactors such that transducer wheels of the plurality of transducer wheels interior to the outermost transducer wheels do not correspond to respective impactors.

In some aspects, the techniques described herein relate to a payload, wherein multiple ones of the plurality of transducer wheels at different horizontal positions along the horizontal axis and at different distances from one of the plurality of impactors sense waves from the inspection surface produced by the one of the plurality of impactors, and wherein the different distances correlate to respective depths of inspection.

In some aspects, the techniques described herein relate to a payload, wherein each of the plurality of transducer wheels includes a plurality of transducers within a material of the transducer wheel.

In some aspects, the techniques described herein relate to a payload, further including: a plurality of transducer wheel assemblies each structured to maintain a fixed position between one of at least some of the plurality of transducer wheels and a corresponding respective one of the plurality of impactors.

In some aspects, the techniques described herein relate to a payload, wherein the horizontal axis is orthogonal to the direction of travel of the inspection robot.

Any improvements, benefits, or the like, as set forth foregoing, are non-limiting examples. Any particular benefit may be present in certain embodiments, and not present in another embodiment. Further, additional benefits and/or improvements may be relevant to listed embodiments, or other embodiments.

### BRIEF DESCRIPTION OF THE FIGURES

FIG. 1 schematically depicts an example payload and a portion of an inspection robot according to example embodiments.

FIG. 2 schematically depicts an example sensing assembly for a payload of an inspection robot according to example embodiments.

FIG. 3 schematically depicts an example sensing assembly for a payload of an inspection robot according to example embodiments.

FIG. 4 depicts a top view of an inspection robot with a payload according to example embodiments.

FIGS. 5A and 5B depict perspective and side views of an inspection robot with a raised payload according to example embodiments.

FIGS. 6A and 6B depict perspective and side views of an inspection robot with a lowered payload according to example embodiments.

FIG. 7 depicts a transducer wheel and an impactor in a transducer wheel assembly according to example embodiments.

FIG. 8 schematically depicts an example cutaway view of an example sensing entity including a transducer within a transducer wheel.

FIG. 9 schematically depicts a transducer wheel on an inspection surface according to example embodiments.

FIG. 10 schematically depicts a transducer wheel on an inspection surface according to example embodiments.

FIG. 11 schematically depicts a transducer wheel on an inspection surface according to example embodiments.

FIG. 12 schematically depicts a transducer wheel on an inspection surface according to example embodiments.

FIG. 13 schematically depicts a configuration of an inspection robot with a payload including a sensing assembly according to example embodiments.

FIG. 14 schematically depicts a configuration of an inspection robot with a payload including sensing assemblies according to example embodiments.

FIG. 15 schematically depicts a configuration of an inspection robot with a payload including sensing assemblies according to example embodiments.

FIG. 16 schematically depicts a configuration of an inspection robot with a payload including sensing assemblies according to example embodiments.

FIGS. 17A and 17B schematically depict a sensing assembly with horizontal resolution widths according to example embodiments.

FIG. 18 schematically depicts a configuration of an inspection robot with payloads including sensing assemblies according to example embodiments.

FIG. 19 schematically depicts a configuration of an inspection robot with payloads including sensing assemblies according to example embodiments.

FIG. 20 schematically depicts a configuration of an inspection robot with payloads including sensing assemblies according to example embodiments.

### DETAILED DESCRIPTION

For the purposes of promoting an understanding of the principles of the disclosure, reference will now be made to the embodiments illustrated in the drawings and described in the following written specification. It is understood that no limitation to the scope of the disclosure is thereby intended. It is further understood that the present disclosure includes any alterations and modifications to the illustrated embodiments and includes further applications of the principles disclosed herein as would normally occur to one skilled in the art to which this disclosure pertains.

Example embodiments herein include inspection robots that are highly configurable to support a broad range of inspection, surface visualization, surface marking, surface cleaning, and/or surface repair operations. Embodiments herein reference an inspection robot as a baseline term to describe a robot that can support any of these operations, including a subset of these operations, or all of these operations, for clarity of the present description. The specific operations performed may nevertheless not be “inspection” operations in certain configurations and/or while performing certain operations. Similarly, embodiments herein reference an inspection surface as a baseline term to describe a service location, and specifically the portion of the service location that is engaged by the inspection robot. An inspection surface, in certain embodiments, may be a serviced portion of the location, whether the specific service(s) performed include(s) inspection, visualization, marking, cleaning, and/or repair. Example and non-limiting inspection surfaces include, without limitation, surfaces such as: a tank wall; a pipe wall; a surface associated with any industrial process or equipment; a cooling tower; a pressure vessel; a tray or interior feature; and/or a heat transfer tube, wall, pipe, or the like. In certain embodiments, an inspection surface may include a metallic surface and/or a ferrous surface. Example inspected surfaces may include any exterior or interior surface, an elevated surface (e.g., a surface including at least a portion that is at a relevant height for fall protection considerations), and/or a confined space (e.g., a surface including at least a portion that would be considered a confined space).

In certain embodiments, an operation may be understood to be an inspection operation for one purpose, but another type of operation for another purpose (e.g., a visualization operation of the surface may be understood to be an inspection operation, but may additionally or alternatively be a preparatory operation, a confirmation operation, etc., which may depend upon the entity describing the operation, whether any anomalies and/or features are detected during the operation, etc.). The specific terminology utilized for an operation is not limiting to the present disclosure, and “inspection operations” or similar terminology utilized herein should be understood to include any service operations, performable by inspection robots set forth herein, at a service location.

Example embodiments utilize modular components that allow for rapid configuration, and/or on-site configuration, for particular operation(s). Further, embodiments herein allow for on-site follow-up inspections, and/or engineering an additional inspection, repair, and/or marking operation on-site within a single service trip to the service location. Example operations utilize sensors or other components (e.g., visualization, cleaning, marking, and/or repair components) that have a wide range of various aspects to support operations, such as: generated and/or collected data rates; data types; required power for operation; provision of supporting fluids such as couplant, cleaning fluids, marking fluids, and/or fluids utilized in repair operations; surface motive engagement assemblies; locating assemblies (e.g., to determine where the inspection robot is on a surface, determination of absolute position, direction, and/or speed of the inspection robot, and/or associating any of these with inspection data and/or supporting data such as pictures, identified obstacles, or the like); power and/or actuating control of supporting assemblies to position the inspection robot and/or portions thereof in a controllable and confirmable manner on the inspection surface; supporting processing for inspection operations, for example onboard processing to interpret raw sensor data into detected conditions of the inspection surface; and/or external communications to/from a base station, operator computing device, and/or cloud server, with communications including data, calibrations, status (e.g., of the inspection robot, the inspection surface, and/or operation level communications such as inspection coverage, progression, stage, etc.), and/or control. The complexity and variety of these supporting aspects present significant challenges to providing an inspection robot that can support a wide variety of operations. Embodiments herein support a wide range of potential applications, with an inspection robot that can be reconfigured by changing a small number of components (or modules) with limited and simplified interfaces. Embodiments herein support inspection robots that can be reconfigured with a small number of tools (e.g., a single wrench of a selected size), and/or in a challenging environment (e.g., in the field rather than in a shop, service location, and/or manufacturing facility, including in an environment with high humidity, dust, mud, rain, etc.), with high confidence that the re-configured inspection robot will be immediately operational without testing and/or with only limited testing (e.g., testing basic functionality from a base station, connected laptop, mobile application in communication with the inspection robot, etc.). Accordingly, embodiments herein support the capability to perform a broader range of services on a broader range of applications, with a single inspection robot and group of modules, than previously known, with significant reductions in costs to configure, reductions in turnaround time to prepare for operations, and/or to respond to conditions that are determined at the service location (e.g., where the determination is made upon visual inspection of the location, according to inspection operations performed at the service location, and/or determined en route to the location—for example reducing the time between a request for service and arrival at the service location by a service operator). Embodiments herein have selected modularity aspects—for example the content and distribution of specific modules-that are selected to support these capabilities and to meet consequent multiple competing goals, for example between: type and/or capability of operations supported; limiting interfaces that are exposed, broken, and/or re-connected during reconfiguration operations; providing a physical footprint that is appropriate for a range of applications and/or inspection surfaces; and/or capability to provide a number of modules within a selected space (e.g., a service truck, pickup bed, flat bed, service van, etc.) to provide a commercially valuable range of capabilities to meet service needs at a selected service location and/or group of service locations.

An example inspection robot may include a core module capable to interface with a number of supporting modules which, when coupled with the core module, provide a completed inspection robot having the selected capability to perform inspection operations. The example core module provides power for operations of the inspection robot, which may include providing power through a tether to a base station, but which may additionally or alternatively include a battery having sufficient energy storage to support a typical inspection operation, and/or to support a selected range of inspection operations (e.g., considering power consumption during operations, the duration of operations, any margin to support uncertainty of inspection operations (e.g., uncertainty of duration and/or power consumption), and/or any power reserve (e.g., preserving sufficient power to return to a base location from any position on the inspection surface). In a non-limiting example, the core module includes a 600 W power supply tethered to a base station, which supports a wide variety of commercially valuable operations for a number of sensor and/or other component configurations. In certain embodiments, the core module mounts on an inspection robot base, which includes the substrate of the inspection robot to provide structural support for the other components of the inspection robot. In certain embodiments, the core module may be considered as a part of the inspection robot base, and/or the inspection robot base may be considered as a part of the core module. In certain embodiments, the core module is swappable to support different capabilities for other modules (e.g., distinct localization modules, DAQ modules, or the like), to support different power ratings for the inspection robot, or the like. In certain embodiments, the core module is universal, for example where the core module is not changed out separately from the inspection robot base. The utilization of a core module allows for other modules to be changed with limited interface adjustments, for example by engaging or disengaging a single connector and/or a limited number of physical support connectors (e.g., screws, bolts, mounting points, quick connectors, etc.), without exposing interior aspects of either the core module (e.g., wires, printed circuit boards, memory chips, power converters, etc.) or the engaged modules (e.g., localization, DAQ, and/or peripheral), reducing the complexity of configuration, and limiting exposure of the modules to environmental intrusion and/or physical damage. An example core module supports communication busses (e.g., ethernet, CAN, and/or I2C), support for a selected number of actuators (e.g., four actuators to support drive modules), and coupling to the base station.

The example core module includes interfaces for mounting three supporting modules thereon. In an example, the three supporting modules include a localization module, a data acquisition module, and a peripheral module. The example modules support a large range of available capabilities for the inspection robot, and are configured to simplify changing out a minimum number of components, with logical breakpoints for selected capability groups, to support high configurability as set forth herein. The example core module includes an electrical/communicative interface for each of the supported modules, and a physical coupling interface for each of the supported modules. In certain embodiments, the physical coupling interface for one or more of the supporting modules, or all of the supporting modules, is keyed to ensure that the supporting module is installed properly. In certain embodiments, supporting modules that are likely to be swapped at the service location, or at a location with minimal facilities, are keyed. In certain embodiments, each of the supporting modules are keyed.

The example localization module includes components that support localization operations of the inspection robot, which may be selected according to the localization requirements of the inspection operations, and/or according to the supporting infrastructure available at the service location. For example, the localization module may include one or more sub-components such as a laser rangefinder, a prism based locator (e.g., a prism on the localization module, and/or a that determines the position of the inspection robot with one or more positioned prisms at the service location), an accelerometer, a gyroscope, a GPS locator device, another locator device (e.g., utilizing WiFi location), or the like. In certain embodiments, localization operations of the inspection robot may be performed utilizing other components of the inspection robot apart from the localization module—for example utilizing a camera associated with the peripheral module and/or utilizing an encoder associated with a drive module and/or a payload of the inspection robot. Certain considerations for determining which components are to be included on a localization module, if present, include the availability of supporting localization infrastructure at the service location (e.g., the availability of located WiFi devices, GPS availability, appropriate locations for positioning of prism(s) and/or rangefinders, and/or the availability of features that can be located and/or evaluated with a camera). Accordingly, the inclusion of a modular localizing component (e.g., the location module) supports rapid reconfiguration of an inspection robot to perform localization operations for a variety of service locations.

The example DAQ module includes data acquisition, processing, and/or communication components to support a selected payload of the inspection robot. For example, distinct sensor suites (e.g., ultra-sonic (UT) sensors, electro-magnetic (EM) sensors, temperature sensors, magnetic flux leakage, visual inspection payloads, profilometers, sonic sensors, etc.) utilize significantly distinct data rates, types of data, supporting data processing, command traffic (e.g., command of sensing operations, fault code traffic, diagnostic traffic, etc.), supported network types (e.g., ethernet, CAN, I2C, etc.), or the like, where utilization of a distinct DAQ module for different sensor suites to allow for quick changes of capability without requiring a software change, communication protocol, I/O changes, or the like that would otherwise be required, for example, to utilize a single generalized DAQ component to provide similar range of capabilities. The various versions of a DAQ module utilize a same interface to the core module to support the full range of DAQ capabilities for the various sensor suites supported by the inspection robot.

The example peripheral module includes interfaces to a payload for the inspection robot, for example to operate associated actuators with the payload (e.g., an actuator to perform rastering operations, to provide selected downforce to the payload, to operate a sprayer for marking and/or cleaning, to operate a repair actuator such as a welder, adhesive dispenser, a laser ablation device, surface preparation device, an induction coating removal device, a couplant flow control valve and/or pump, etc.). The example peripheral module further includes selected supporting components for the inspection robot—for example a camera—and/or includes interfaces to such components (e.g., where a camera is provided on the payload). The utilization of a peripheral module allows for flexible support for a number of components, dividing the responsibility between the relatively consistent operations performed to support sensing (e.g., via the DAQ module), localization operations (e.g., via the localization module), and flexible operations for peripheral components (e.g., via the peripheral module). The division of responsibilities between the localization module, DAQ module, and/or the peripheral module is a non-limiting example, and provides for a logical grouping of responsibilities that are capable to support a wide range of commercial applications.

Certain aspects of the inspection robot, for example interfaces with the payload, may interface with multiple ones of the supporting modules. For example, sensor data and control for sensors of the payload are provided through the DAQ module, and payload actuator control of the payload is provided through the peripheral module. Stated differently, the organization of modules in the depicted example is a functional organization. In certain embodiments, a different organization of modules may be provided, for example one supporting module may interact with the payload, including sensing and actuating. Additionally or alternatively, a component on one supporting module may support operations generally associated with another supporting module—for example a camera associated with a peripheral module may be considered as an inspection sensor for certain inspection robots and/or inspection operations (and/or another camera associated with the DAQ module may be present for certain embodiments).

An example embodiment further includes a number of drive modules configured to provide motive power and control of the inspection robot on the inspection surface. In certain embodiments, the drive modules are directly coupled to interfaces on the core module. Additionally or alternatively, drive modules may interface with and/or be controlled by another module, for example the peripheral module. The example drive modules are depicted as magnetic hub-based wheels, but any type of drive module and/or motive movement and/or control may be utilized.

An example core module may be mounted on an inspection robot base, separated from the inspection robot base, and may include an electrical interface with keyed support connections for a supporting module.

An example assembled inspection robot may include a localization module, DAQ module, and peripheral module mounted on a core module. The example may also include a payload having a rastering actuator (raster arm payload), for example allowing for inspection, repair, welding, and/or marking operations that can be positioned across the horizontal width of the inspection robot (and/or within the rastering range, which may be greater or smaller than the horizontal width of the inspection robot). The example may include an example payload mounting location for the payload—for example with a mounting location at the front of the inspection robot. The inspection robot may support any type of payload that can be mounted on the inspection robot, with control and data operations for the payload provided by the supporting modules as set forth herein.

An example suspension system for drive modules herein may provide for coordinated movement of the individual elements of the drive module (and/or for each drive module, depending upon whether each wheel and/or motor is considered as an element of the drive module, or as a separate drive module). In the example embodiments, the drive module(s) is mounted physically to the inspection robot base, and interfaces with and is controlled by the core module.

The example drive module(s) include diametrical cooling fins, which are thermally coupled to drive motors positioned within the drive module, and provide for passive cooling of the drive modules. In certain embodiments, for example where a payload includes UT sensors having a couplant provided to the inspection robot for supporting operations of the UT sensors to acoustically couple to the inspection surface, it may be desirable to utilize the couplant for cooling of the drive modules and/or heat generating components within the other modules (e.g., PCBs, power converters, etc. within the core module, DAQ module, localization module, and/or peripheral module). In certain embodiments, performing cooling without utilizing available couplant supports the modularity, flexibility, and/or configurability of the inspection robot—for example providing an inspection robot where sufficient cooling is performed passively, where the inspection robot performs for payloads either with or without available couplant. In certain embodiments, for example where couplant or any other fluids (e.g., cleaning, surface preparation, and/or repair fluids) are provided to the inspection robot, such fluids are provided directly to the utilizing component (e.g., the payload of the inspection robot), and are not used secondarily for module support. In certain embodiments, supporting operations for managing such fluids may be nevertheless performed by one or more modules, for example with a flow control valve or pump operated by the peripheral module. The example inspection robot may further include a dual-purpose port, provided on the core module in the example, that allows for leak testing and provides a place to engage a desiccant that is operationally coupled to the core module (e.g., to protect components, such as PCBs and/or power converters, from humidity or the like). In certain embodiments, leak testing and/or desiccant holding functions may be performed utilizing separate ports, and/or omitted. The core module may further include cooling fins, for example to support passive cooling of the core module. The example cooling fins, for both the drive module(s) and the core module, are geometrically positioned to support passive cooling on either a horizontal or vertical inspection surface, further supporting flexible capability for the inspection robot. In certain embodiments, any motors, actuators, or other heat generating components of the inspection robot are configured to perform with passive cooling, including thermally coupling heat generating components with heat rejection components, providing cooling fins associated with supporting modules, payloads, or the like.

An example encoder couples to the inspection robot base and/or core module, including physical mounting and/or electro-mechanical mounting. The example encoder supports position determination of the inspection robot, and/or is utilized in control of the drive modules. In certain embodiments, the encoder includes serrations that are configured to support operations of the encoder without slipping, and without marking or scratching the inspection surface, for example if side-to-side movement of the encoder occurs while engaged with the inspection surface. The example inspection robot further includes a tether coupling that is configurable, for example by swapping out the core module. In certain embodiments, the tether connection is split, for example with fluids bypassing the core module and passing directly to the utilizing component, for example to the payload. In certain embodiments, the tether includes power, communication, and/or electrical connections directly coupled to the core module, where a single tether supports a wide range of applications and does not need to be configured for the particular application.

An example inspection robot may include supporting modules in an engaged position and in a disengaged position, which may include operations performed to reconfigure the inspection robot to change capabilities, to prepare for specific operations, or the like. The locational module and DAQ module may be disengaged, as a non-limiting example. For example, a peripheral module may not be changed during a given re-configuration operation. In another example, the peripheral module may be at an intermediate stage of a re-configuration operation, where the peripheral module has already been swapped, or will be swapped at a later time.

An example inspection robot includes a rastering payload, a localization module with a locating prism mounted thereon, a DAQ module, and a peripheral module. Another example inspection robot may include another example localization module having a range finder mounted on a rotatable actuator. Another example inspection robot may be either at an intermediate configuration stage (e.g., before the DAQ module and localization module are engaged), and/or in a configuration where a DAQ module and localization module are not needed for the planned inspection operations. The example inspection robot includes an alternate assembly for the drive module, with a tracked drive module depicted in the example. The example drive module may include magnetic elements, for example where an inspection surface includes a ferrous surface.

An example inspection robot may include an encoder and drive modules. In some examples, an example inspection robot may include diametrical fins on a drive module.

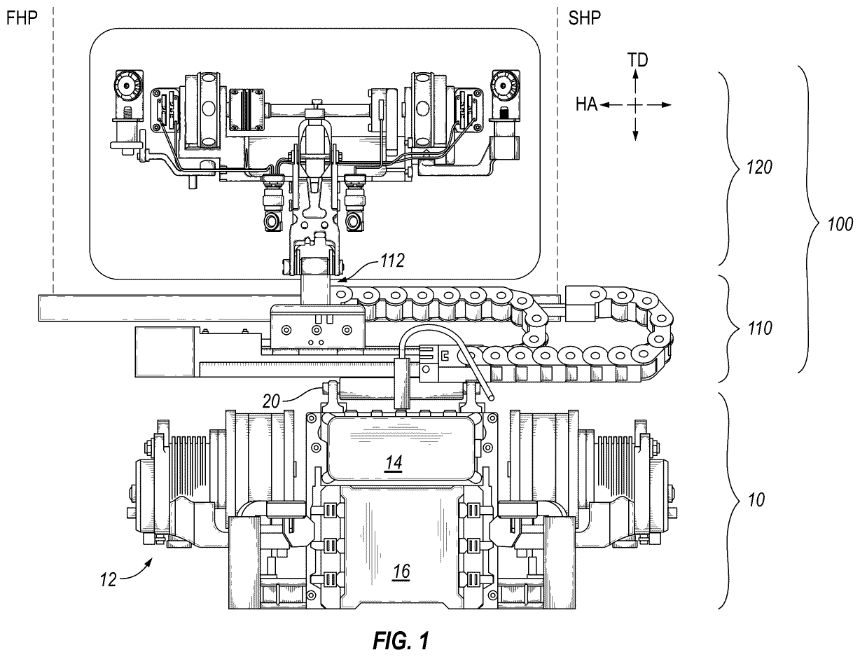

Referencing FIG. 1, an example payload 100 and a portion of an inspection robot 10 according to an example embodiment are schematically depicted. The example payload 100 may include at least one sensing assembly 120 (e.g., at least one concrete sensing assembly) and an example payload mount 110. However, embodiments are not limited thereto, and in some examples, the payload mount 110 may be considered separate from payload 100. The example payload 100 may be usable with any inspection robot 10 as may be set forth in example embodiments throughout the present disclosure.

In some examples, the payload mount 110 may be mechanically connected to an inspection robot 10 through a payload mounting 20. In an example, the payload mounting may include a pivot joint between the inspection robot 10 and payload mount 110, which may be adjusted and fixed in a selected position. The payload mounting 20 may, for example, be at a mounting location at the front of the inspection robot 10. In some examples, the inspection robot 10 may include such components as a drive module 12, a peripheral module 14, a DAQ module 16, and one or more controllers, some of which may be included in the modules, among other components. A controller as described herein may include and/or embody one or more processors and/or other circuitry.

In some embodiments, the payload mount 110 may include a rastering actuator 112 having a rastering arm and capable to move the payload horizontally (e.g., along a horizontal axis HA), for example to extend the horizontal inspection width available to the inspection robot 10, thereby extending the available surface area for inspection of the inspection robot 10 in a given pass of the inspection robot 10. The example of FIG. 1 depicts a forward portion of the inspection robot 10 for context, and depicts the payload mounting 20 consistent with example embodiments described herein.

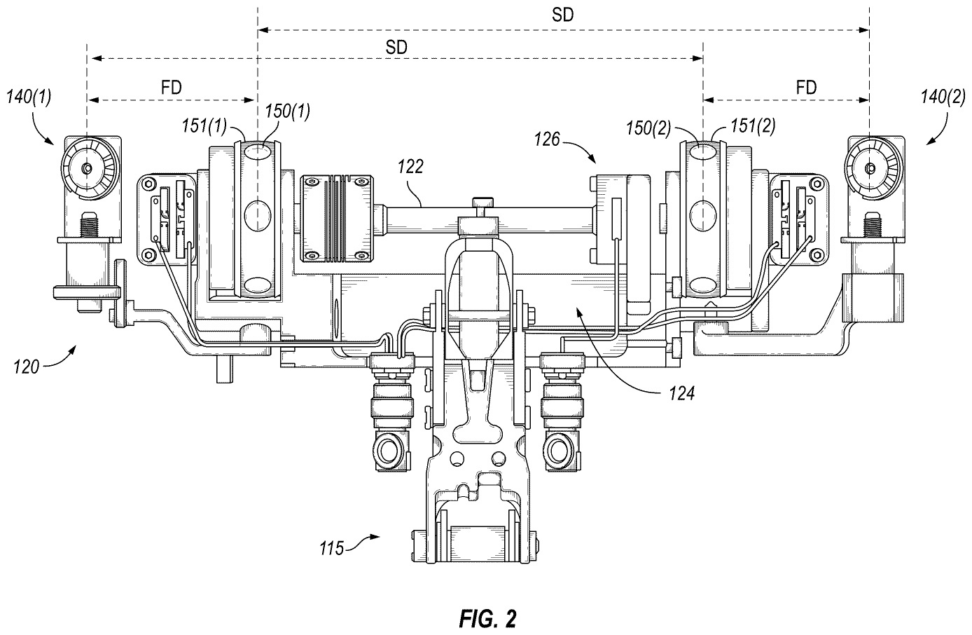

Referencing FIG. 2, schematically depicted is an example sensing assembly 120 (which, in example embodiments, may be referred to as a concrete sensing assembly without constraining it to the sensing of only certain materials) of an example payload 100 for an inspection robot 10 according to example embodiments. The example embodiment of FIG. 2 may be consistent with aspects of the example embodiment depicted in FIG. 1. The example payload 100 may be an acoustic sensing payload, utilized in an example inspection operation to inspect concrete (or cement, which may be referenced as concrete herein) placement, for example with regard to a concrete supported tank, and/or for example with a metal surface (e.g., a steel wall) having an associated concrete support. Example inspection operations may be performed to determine proper placement and/or characteristics of the concrete—for example, bonding of the concrete with the metal surface, detection of voids within the concrete, and/or determination of a density and/or porosity of the concrete (e.g., to ensure that a designed density and/or porosity of the concrete has been achieved). In certain embodiments, the inspection robot 10 may operate on a metal surface (e.g., a steel plate, tank wall, tank floor, etc.), and perform concrete inspection on a concrete substrate positioned on an opposite side of the metal surface from the inspection robot 10. In certain embodiments, the inspection robot 10 may operate directly on a concrete surface, which may be fully exposed, treated, coated, and/or painted.

A sensing assembly 120 of example payload 100 according to example embodiments may include at least one or a plurality of impactors 140 (e.g., energy generators, such as including a piezoelectric actuator) and associated pressure sensors, such as transducers 150. In example embodiments, distances between the impactors 140 and transducers 150 may be selected for appropriate inspection configurations (e.g., via one or more coupling shafts 122) such as a depth of inspection. The transducers 150 may provide data that is processed to provide analysis of compression wave returns, shear wave returns, and/or surface wave returns, which can be processed to determine bonding characteristics, the presence of voids, and/or acoustic characteristics of the concrete (e.g., by determining the associated speed of sound in the substrate of the concrete). In certain embodiments, various potential defects in the concrete may be determined by inspection operations, such as: a void (e.g., air, water, soil, low density concrete, etc.), a crack, a debonded portion, a delaminated portion, a honeycombed portion, corrosion (e.g., of rebar, shear anchors, etc.), a cold joint, soil chemical corrosion (e.g., of concrete), and/or rebar spacing.

A sensing assembly 120 of an example payload 100 according to example embodiments may include a coupling shaft 122 that supports transducer 150/impactor 140 pairs, enforces a designed distance between the transducer 150/impactor 140 pairs (such as deigned distances between each transducer 150/impactor 140 and other transducers 150/impactors 140), and/or maintains a selected angular position (e.g., a selected offset) between the transducers 150. While example embodiments described herein may describe a sensing assembly 120 in its singular form, example embodiments are not limited to a single sensing assembly 120, and indeed, as discussed herein, some embodiments may include a plurality of sensing assemblies 120.

In example embodiments, the sensing assembly 120 of example payload 100 may include an encoder 126 to determine and/or confirm the rotating position of elements of the transducers 150. For example, in embodiments including transducer wheels 151 each with a plurality of transducers 150, the encoder 126 may be used by the inspection robot 10 (e.g., by a controller of the inspection robot 10) to determine a position and/or phase of transducers 150 on the transducer wheel 151.

In example embodiments, the sensing assembly 120 of example payload 100 may include a chassis body 124 that serves as a mounting substrate for a sensing package including the transducers 150 and impactors 140, and which provides for mounting of the payload 100 on the inspection robot 10—for example, via payload mount 110. In the example of FIGS. 1 and 2, a coupling arm 115 may be utilized to mount the payload 100 (e.g., the sensor assembly 120) to the inspection robot 10 and/or the payload mount 110, and may provide a roll and pitch degree of freedom to the payload 100 and/or sensing assembly 120, such as to ensure appropriate coupling of the sensing package with the inspection surface, to allow traversal of obstacles, etc.

In certain embodiments, the payload 100 may have an applied downforce, either passively (e.g., using a biasing spring, selected weight, etc.) and/or actively (e.g., with a linear and/or rotating actuator, which may apply a selected downforce, and/or utilized to selectively raise the payload from the inspection surface). Active downforce may be controller by a controller such as included in the inspection robot 10. Indeed, in certain embodiments, communications and/or control with the impactors 140, transducers 150, and/or encoder 126 may be performed by a controller of the inspection robot 10, such as, and/or in a DAQ module 16 and/or peripheral module 14. For example, in certain embodiments, for example where active downforce and/or lifting actuator(s) are present, such operations may be controlled by the peripheral module 14, such as a controller in the peripheral module 14.

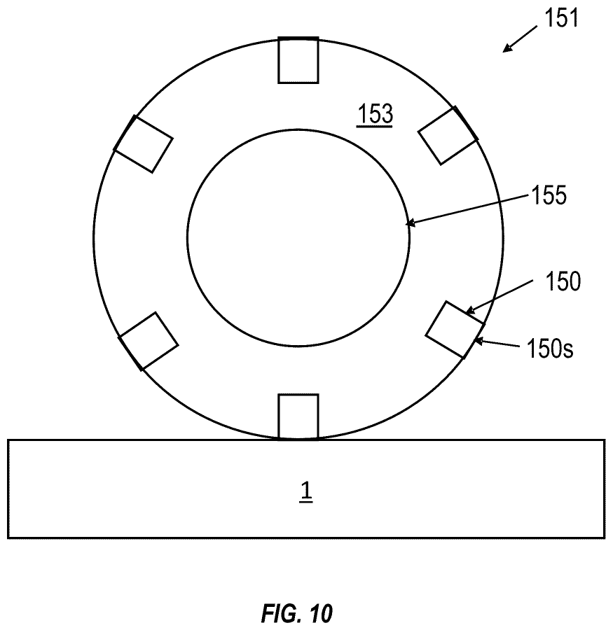

As illustrated in the example embodiment of FIG. 2, and as may be described in further detail with reference to FIGS. 10-12, the transducers 150 may be provided as circumferentially distributed transducers 150 that sequentially engage the inspection surface on one or more transducer wheels 151. The one or more transducer wheels 151 may serve as a wheel mount for the transducers 150, and may rotate in response to movement of the inspection robot 10 along the inspection surface. As discussed herein, the number and spacing of the transducers 150 on a transducer wheel 151 may be selectable.

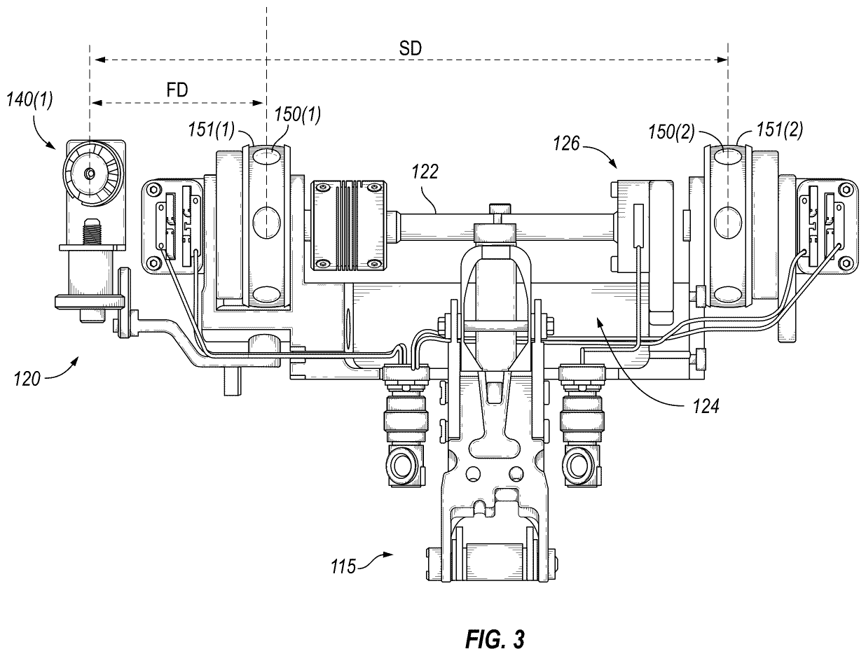

Referencing FIG. 3, an overhead view of an example concrete sensing assembly 120 of a payload 100 is depicted. In the example of FIG. 3, a single impactor 140 and two transducer wheels 151 (which may be alternatively referred to herein as transducer assemblies) are depicted. The example of FIG. 3 is consistent with a payload 100 for an inspection robot 10 during a configuration change, for example during positioning and/or replacement of the second impactor, and/or with a sensing package utilizing a single impactor 140—for example, utilizing the closer transducer 150 (e.g., closer transducer wheel 151(1)) for compression and/or shear wave analysis, and the distant transducer 150 (e.g., distant transducer wheel 151(2)) in shear wave analysis. Embodiments herein reference compression wave analysis and/or shear wave analysis for clarity of the description, but any type of analysis may be utilized, including for example surface wave analysis and/or spectral analysis of surface waves. Relative to an embodiment utilizing two impactors 140, inspection of the entire surface for compression analysis may take slightly longer, with extended rastering operations to provide the same coverage. In certain embodiments, reduced cost and/or data/control support for a single impactor 140 may be a worthwhile tradeoff relative to the decrease in inspection speed relative to the two impactor embodiment.

An example compression analysis utilizing data provided by a sensing assembly 120 may be capable to detect the presence of a void in the concrete, for example comparing the signal analysis from illustrative data for acceptable concrete with illustrative data for concrete having a void. Example shear analysis may be capable to characterize a void in the concrete, for example including a characterization of the depth and width of the void. Additionally, the shear wave analysis may be capable of characterizing the presence of a void even behind poorly bonded concrete (e.g., comparing to illustrative data signatures).

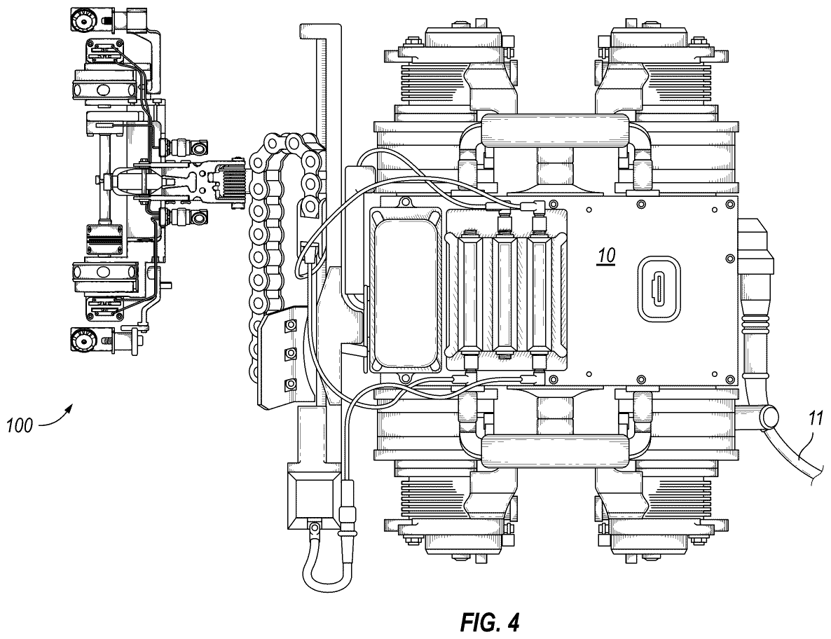

Referencing FIG. 4, a top view of an inspection robot 10 connected to a payload 100 consistent with example embodiments is depicted. A tether 11 is depicted in the example of FIG. 4, which is optional and which may supply power, communications, and/or couplant to the inspection robot 10 from the appropriate components, such as a base station (e.g., an operator laptop, mobile device, communication device to a cloud server), a power supply, and/or a couplant supply.

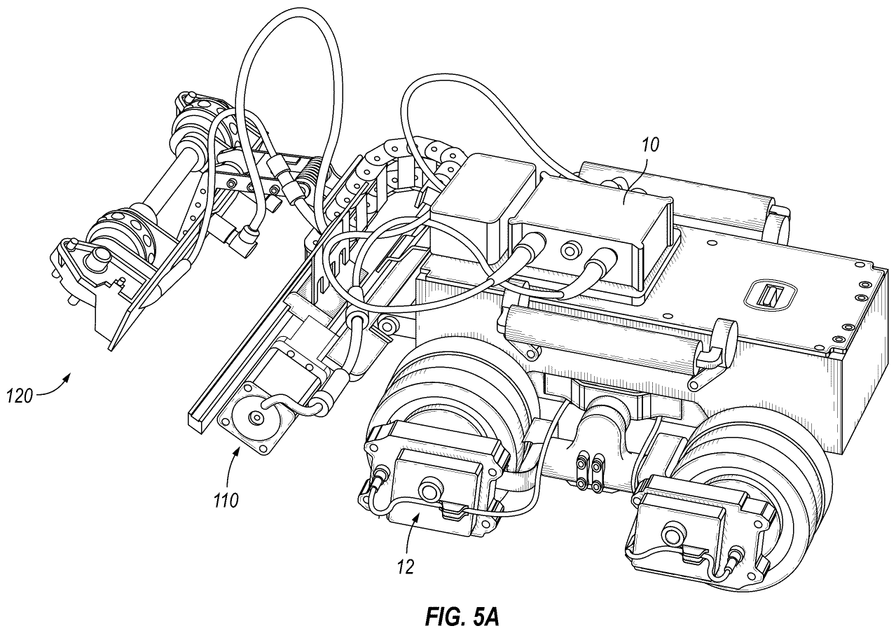

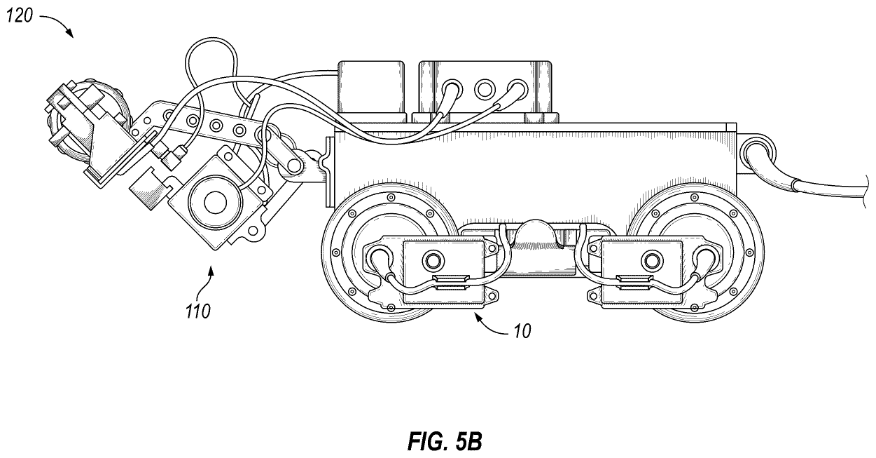

Referencing FIG. 5A, a non-limiting example of an inspection robot 10 positioned on an inspection surface is schematically depicted. The example inspection robot 10 includes a payload 100 having a concrete sensing assembly 120 (e.g., impactor(s) 140 and/or transducer(s) 151 in a selected arrangement) and a payload mount 110 that couples the payload (e.g., the sensing assembly 120) to the inspection robot 10 and provides various actuating functions such as rastering of the concrete sensing assembly 120, raising or lowering the concrete sensing assembly 120, and/or providing selected downforce to the concrete sensing assembly 120. FIG. 5A illustrates the payload 100 including a concrete sensing assembly in a raised position—for example, utilized to traverse obstacles, to move the inspection robot from one position to another on the inspection surface, or the like.

The payload 100 illustrated in the example of FIG. 5A may include the concrete sensing assembly 120 and the supporting arm (not labeled), which may be part of the payload mount 110. Furthermore, in certain embodiments, one or more aspects of the inspection robot 10 may be a part of the payload 100—for example, where a power supply, data acquisition hardware, and/or an electronic card is included on the inspection robot 10 to support a particular payload 100. In certain embodiments, those supporting elements on the inspection robot 10 that are changed, confirmed, calibrated, or otherwise configured in response to the installation of a particular payload may be considered as a part of the payload 100 itself, and/or may be considered as a part of the remainder of the inspection robot 10 (e.g., the rest of the inspection robot 10 that is not the payload itself 100). In the example of FIG. 5A, one or more drive modules 12 may provide for motive power and control of the inspection robot 10. Meanwhile, FIG. 5B illustrates a side view of the payload 100 and inspection robot 10 of FIG. 5A, including said payload 100 being in a raised position from the inspection surface.

Indeed, with reference to FIGS. 1 and 2, the example payload 100 for an inspection robot 10 to inspect an inspection surface as illustrated in FIGS. 5A-5B may include an example concrete sensing assembly 120 including a first impactor 140(1) at a first end, a first near transducer 150(1) (which, in some embodiments, may be part of a first transducer wheel 151(1)) at a first distance FD from the first impactor 140(1), and a second far transducer 150(2) (which, in some embodiments, may be part of a second transducer wheel 151(2)) at a second distance SD from the first impactor, where the second distance SD is greater than the first distance FD. Additionally, in some embodiments, the concrete sensing assembly 120 may include a second impactor 140(2) positioned at a second end, where the second end is opposite to the first end, the second far transducer 150(2) is at the first distance FD from the second impactor 140(2), and the first near transducer 150(1) is at the second distance SD from the second impactor 140(2). As described herein, a distance between impactor 140(1)/transducer wheel 151(1) and impactor 140(2)/transducer wheel 151(2) may be maintained by a coupling shaft 122. Consistent with impactors 140 as described herein, the first impactor 140(1) and/or second impactor 140(2) may each include a piezoelectric material to provide an impact against the inspection surface when the piezoelectric material is provided with an electric charge.

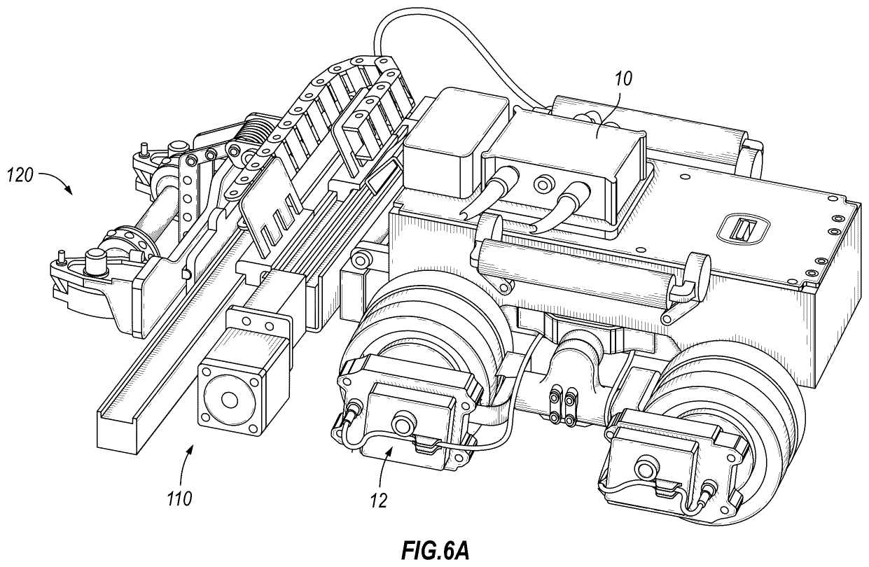

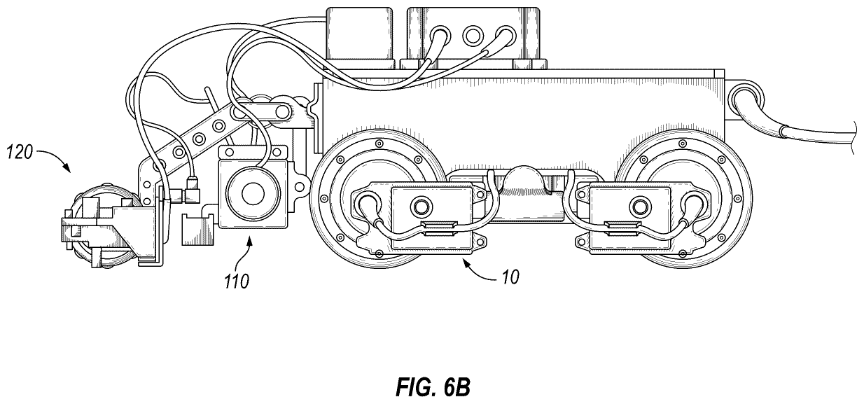

The example payload 100 may include a payload mount 110 structured to couple the concrete sensing assembly 120 to the inspection robot 10, and to move the concrete sensing assembly 120 between at least a first lowered position and a second raised position. As described above, FIGS. 5A-5B illustrate the concrete sensing assembly 120 in the second raised position. When the concrete sensing assembly 120 is in the second raised position, the first impactor 140(1) may be spaced apart from the inspection surface. Meanwhile, FIGS. 6A-6B illustrate the example concrete sensing assembly 120 of FIGS. 5A-5B with the payload 100 including the concrete sensing assembly 120 in the first lowered position-e.g., against the inspection surface such that the impactor(s) 140 and transducer(s) 150 make contact with the inspection surface. In this position, the concrete sensing assembly 120 may be utilized to inspect the inspection surface. As an example, referring to FIG. 2, when the example concrete sensing assembly 120 is in the first lowered position, the first impactor 140(1) is in contact with the inspection surface. Indeed, in that example, second impactor 140(2) is also in contact with the inspection surface.

The payload mount 110 may include at least one of a spring and/or actuator to move the concrete sensing assembly 120 between the first lowered position and the second raised position. In some examples, the payload mount 110 may include a downforce structure to provide a selected downforce of the concrete sensing assembly 120 including the first impactor 140(1) against the inspection surface. For example, that downforce structure may include the at least one spring and/or actuator. Additionally and/or alternatively, in some embodiments, the payload mount 110 may include a spring-based structure having one or a plurality of springs to provide a selected downforce of the concrete sensing assembly 120 against the inspection surface when the concrete sensing assembly 120 is in the first lowered position and to provide a selected upforce of the concrete sensing assembly 120 away from the inspection surface when the concrete sensing assembly 120 is in the second raised position. For example, the spring(s) may provide increased tension when the concrete sensing assembly 120 is between the first lowered position and the second raised position, encouraging the concrete sensing assembly to remain in the one of the first lowered position or the second raised position until an adequate force (e.g., as provided via an actuator) overcomes the spring tension and permits the concrete sensing assembly 120 to move to the other of the two positions.

As described herein, a system according to example embodiments may include the payload 100 and inspection robot 10 described above, and the inspection robot 10 may include a controller to instruct the payload mount 110 to move the concrete sensing assembly 120 between the first lowered position and the second raised position. For example, the controller may instruct the payload mount 110 to move the concrete sensing assembly 120 to the second raised position when the inspection robot 10 detects an obstacle or other abnormality on the inspection surface in a direction of travel of the inspection robot 10, or at any time when the inspection robot 10 is not performing an inspection operation to avoid wear and tear on the transducers and impactors.

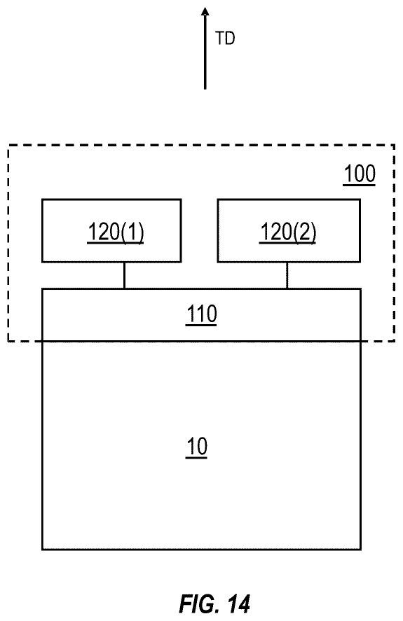

With reference to FIG. 2, in example embodiments, the first near transducer 150(1) may be included in a first near transducer wheel 151(1), and the second far transducer 150(2) may be included in a second far transducer wheel 151(2). Example embodiments are not limited to a single sensing assembly 120, and in examples, the system may further include another concrete sensing assembly 120, which may include at least a third transducer wheel 151 having a third transducer 150. For example, see FIGS. 14-16 and corresponding description. In examples including another concrete sensing assembly 120 with a third transducer 150, the controller (e.g., of the inspection robot 10 although not limited thereto) may instruct the payload mount 110 to move the concrete sensing assembly 120 between the first lowered position and the second raised position to change a sensing phase of at least one of the first near transducer 150(1) or the second far transducer 150(2) relative to the third transducer 150 of the third transducer wheel 151 on the another concrete sensing assembly 120. For example, each of the first, second, and third transducers 150 may be included on respective transducer wheels 151. The first and second transducers 150 may rotate together on their respective transducer wheels 151 owing to their inclusion on a same concrete sensing assembly 120 and simultaneous contact/lack of contact with inspection surface when the concrete sensing assembly 120 is in the first lowered position/second raised position. However, as the third transducer 150 may be on a transducer wheel 151 that is part of a different concrete sensing assembly 120, the third transducer 150 (and indeed, all transducers of its transducer wheel 151) may be operated in or out of phase with the first and second transducers 150 (and indeed, all transducers of their transducer wheels 151). In some embodiments, it may be desirable to operate the transducers out of phase, for example to adjust sampling time windows for transducers (e.g., to reduce potential cross-talk), to adjust the sampled locations on the surface, and/or to cap bandwidth utilization within the system (e.g., between active transducers and a data acquisition component on the inspection robot).

With reference to FIGS. 1 and 2, in example embodiments, the example payload 100 for an inspection robot 10 to inspect an inspection surface as illustrated in FIGS. 5A and 5B may include a concrete sensing assembly 120 including at least one transducer 150 (e.g., as part of at least one transducer wheel 151) and at least one impactor 140, the at least one transducer 150 at a first distance FD from the at least one impactor 140 on a horizontal axis HA to define a first inspection depth and a first horizontal inspection width, where the first horizontal inspection width corresponds to the first distance FD. In example embodiments, the horizontal axis may be orthogonal to a travel direction TD of the inspection robot 10.

The payload 100 may further include a payload mount 110, where the payload mount 110 may be structured to couple the concrete sensing assembly 120 to the inspection robot 10, and which may include rastering actuator 112, which, for example, moves along a horizontal rail. The rastering actuator 112 may be structured to move the concrete sensing assembly 120 (e.g., at a center of the concrete sensing assembly 120) between a first horizontal position FHP and a second horizontal position SHP on the horizontal axis (e.g., along the horizontal rail) to provide a rastered horizontal inspection width that is greater than the first horizontal inspection width.

In some embodiments, the at least one transducer 150 may include a plurality of transducers 150 (e.g., as part of a plurality of transducer wheels 151), and the at least one impactor 140 may include a plurality of impactors 140. For example, with reference to FIG. 2, example embodiments may include a plurality of transducers including transducers 150(1) and 150(2) and a plurality of impactors 140 including impactors 140(1) and 140(2). At least some of the plurality of impactors 140 may correspond to respective ones of the plurality of transducers 150. For example, with reference to FIG. 2, impactor 140(1) may correspond to transducer 150(1) (and, indeed, may correspond to all transducers of transducer wheel 151(1)), and impactor 140(2) may correspond to transducer 150(2) (and, indeed, may correspond to all transducers of transducer wheel 151(2)). Such impactors 140 and transducers 150 may correspond in “pairs” insofar as they are not spaced apart from each other using, e.g., a coupling shaft 122 that supports transducer 150/impactor 140 pairs and enforces a designed distance between pairs as described herein, and/or by their containment in a same wheel assembly.

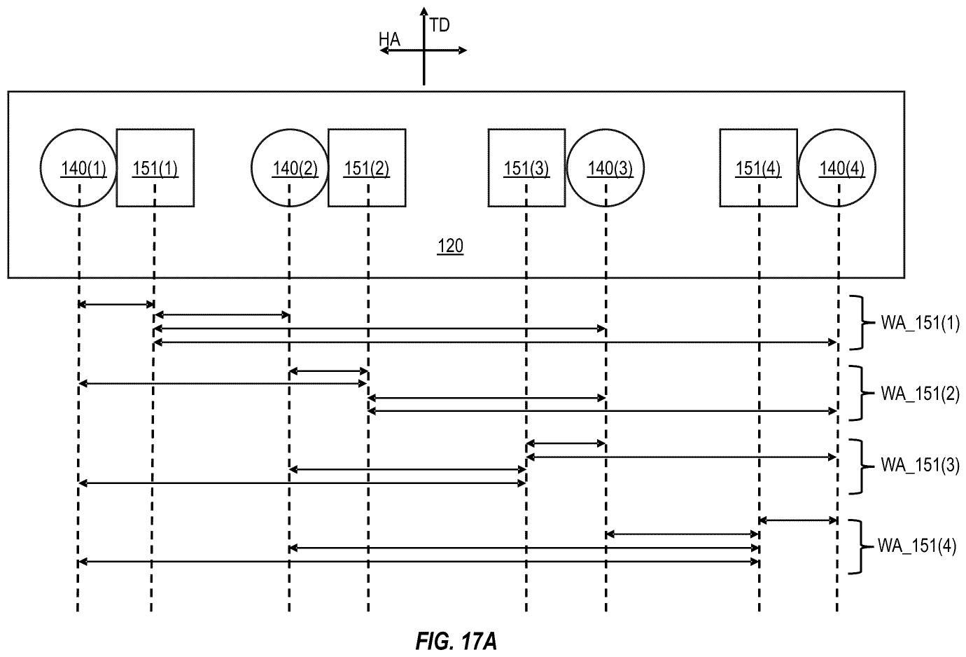

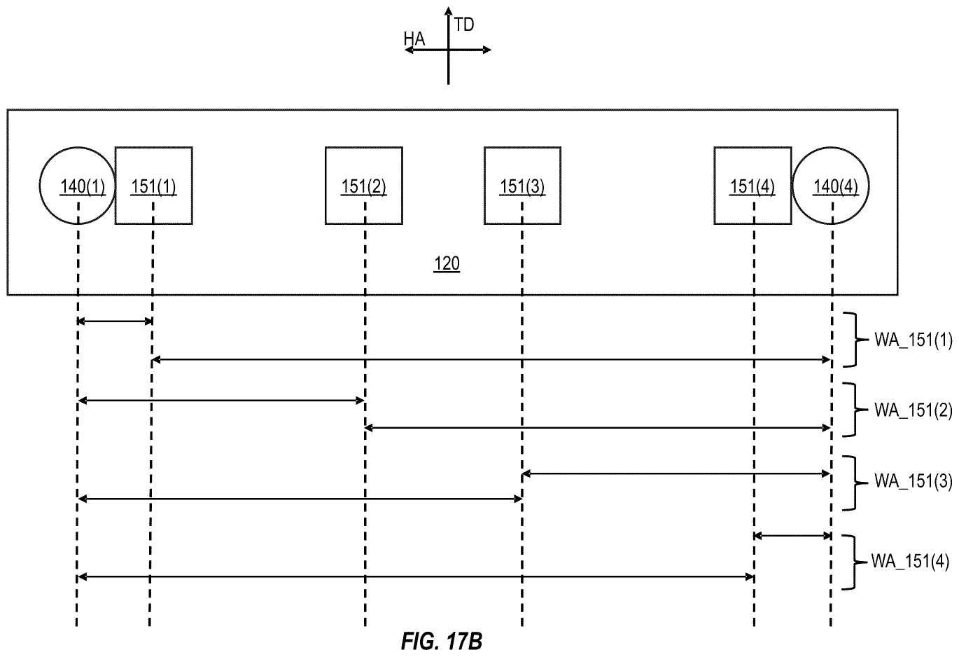

In some embodiments, the plurality of transducers 150 and the plurality of impactors 140 may define a plurality of horizontal inspection widths corresponding to respective distances between the plurality of transducers 150 and the plurality of impactors 140. For example, with reference to FIG. 2, a plurality of horizontal inspection widths may include each horizontal inspection width corresponding to a distance FD or a distance SD. As another example, with reference to FIG. 17A, which shows a schematic overhead view of a plurality of impactors 140 and transducers 150 in pairs as part of a concrete sensing assembly 120 according to example embodiments, horizontal inspection widths may be defined by each arrow indicated at WA_151(1), WA_151(2), WA_151(3), and WA_151(4). Wave analysis may be accordingly be performed based on the sensing of respective transducer wheels 151(1), 151(2), 151(3), and 151(4) using the different corresponding horizontal inspection widths at WA_151(1), WA_151(2), WA_151(3), and WA_151(4).

Also, in some embodiments, not every transducer wheel 151 may include a corresponding impactor 140. As shown by example in FIG. 17B, which is a schematic overhead view of a plurality of impactors 140 and transducers 150 as part of a concrete sensing assembly 120 according to example embodiments, horizontal inspection widths may be defined by the transducer wheels 151 and impactors 140 that do exist in the sensing assembly 120.

With reference again to FIG. 1 as well as FIGS. 17A and 17B, in some embodiments, the rastering actuator 112 may be structured to move the concrete sensing assembly 120 between the first horizontal position FHP and the second horizontal position SHP to provide a plurality of rastered horizontal inspection widths that are greater than the respective plurality of horizontal inspection widths.

In some embodiments, a system may include the payload 100 described above as well as the inspection robot 10, and the inspection robot 10 (or other portion of the system) may include a controller to instruct the rastering actuator 112 to move the concrete sensing assembly 120 in two opposite directions (e.g., along the horizontal axis HA) between the first horizontal position FHP and the second horizontal position SHP. For example, the controller may instruct the rastering actuator 112 to move the concrete sensing assembly 120 in at least one of the two opposite directions when the inspection robot 10 is not moving in an inspection direction (e.g., a travel direction TD). For example, the inspection robot 10 may move a distance in the travel direction TD, pause movement and raster the sensing assembly 120 using the rastering actuator 112 while collecting inspection data from the impactors 140 and transducers 150, then move again in the travel direction TD until it reaches the next position for inspection, where the rastering and inspection process is repeated. However, embodiments are not limited thereto, and in some example embodiments, owing to the relative high speed of inspection versus the lower speed of travel, the sensing assembly 120 may conduct sensing operations (with or without rastering) while the inspection robot 10 is in motion in the travel direction TD.

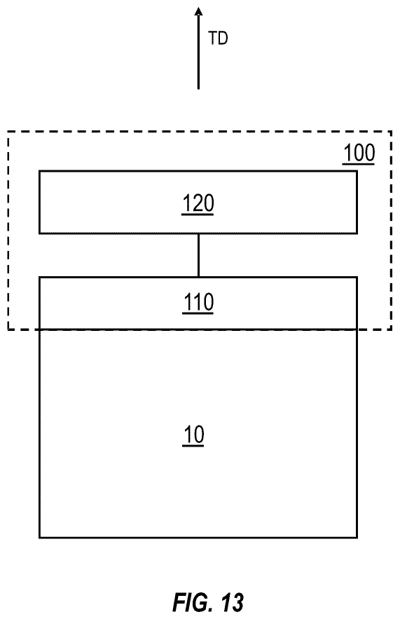

In example embodiments, with reference to FIGS. 13 and 14, which illustrate schematic example configurations of an inspection robot 10 and payload 100 with one or more sensing assemblies 120, a payload 100 for an inspection robot 10 to inspect an inspection surface may include a single sensing assembly 120 as shown by example in FIG. 13, or it may include a first plurality of sensing assemblies 120(1), 120(2) horizontally distributed across the payload relative to a direction of travel TD of the inspection robot 10, as illustrated by example in FIG. 14. At least one of the plurality of concrete sensing assemblies 120(1), 120(2), and in some embodiments, all or some of the plurality of concrete sensing assemblies 120(1), 120(2), may include a impactor 140 and transducer 150 configuration like as described with reference to FIGS. 1 and 2. The payload may include a payload mount 110 structured to couple the concrete sensing assemblies 120(1), 120(2) to the inspection robot 10.

Two sensing assemblies 120 are illustrated for example only; embodiments are not limited thereto and may include more sensing assemblies 120. For example, some embodiments may include three or four sensing assemblies 120 horizontally distributed. Example embodiments with a plurality of sensing assemblies 120 horizontally distributed across the payload 10 may enhance the rate of inspection coverage, and may be combined with rastering operations to provide for rapid inspection of an inspection surface. In certain embodiments, the sensing assemblies 120 may be identical, for example providing greater inspection coverage in a single pass of the inspection robot, and/or may be distinct sensing packages (e.g., utilizing different types of sensors, configurations to inspect different depths of the surface, etc.), allowing for enhanced coverage (e.g., surface area inspected per pass), and/or enhanced detection (e.g., detecting multiple types of features, multiple depths of inspection, etc. per pass).

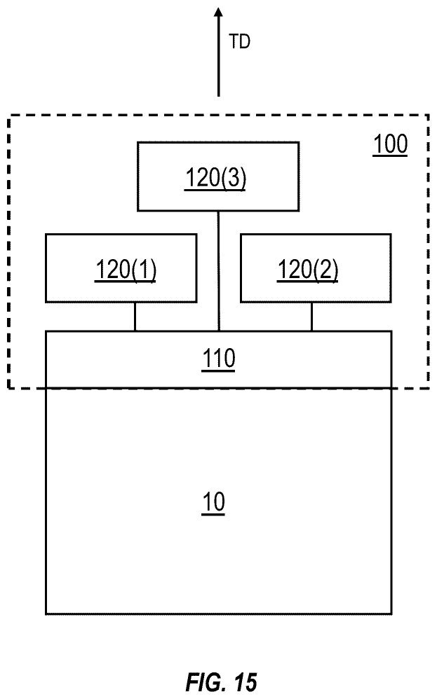

In some embodiments, with refence to FIG. 15, which shows a schematic example configuration of an inspection robot 10 and payload 100 with sensing assemblies 120, the plurality of concrete sensing assemblies 120 may include at least one concrete sensing assembly 120(3) positioned forward of the first plurality of concrete sensing assemblies 120(1), 120(2) relative to the direction of travel TD of the inspection robot 10 to provide increased horizontal resolution of an inspection operation of the inspection surface. For example, as shown in FIG. 15, the at least one concrete sensing assembly 120(3) may be positioned at a gap position to cover a horizontal gap between two or more of the first plurality of concrete sensing assemblies, such as concrete sensing assemblies 120(1), 120(2). In some examples, this at least one concrete sensing assembly 120(3) may be positioned at or near (e.g., approximately at) a horizontal center of the payload 100 to inspect a feature of the inspection surface, such as a weld line or other structural feature. Thus, for example, the at least one concrete sensing assembly 120(3) may include a sensing package that is distinct from a sensing package of at least one of the first plurality of concrete sensing assemblies 120(1), 120(2).

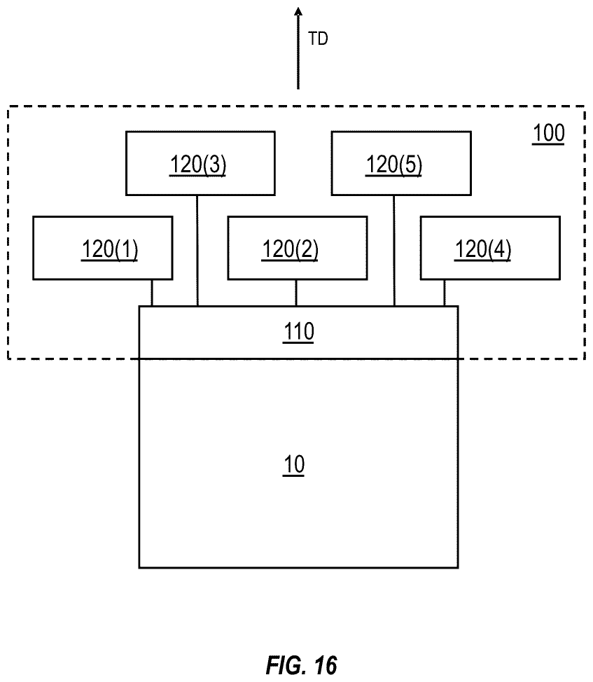

In some embodiments, with reference to FIG. 16, which shows a schematic example configuration of an inspection robot 10 and payload 100 with sensing assemblies 120, the plurality of concrete sensing assemblies may further include a plurality of forward concrete sensing assemblies 120(3), 120(5) positioned forward of the first plurality of concrete assemblies 120(1), 120(2), 120(4) relative to the direction of travel TD of the inspection robot 10. As shown in FIG. 16, the plurality of forward concrete sensing assemblies 120(3), 120(5) may be positioned at respective gap positions to cover horizontal gaps between respective two or more of the first plurality of concrete sensing assemblies 120(1), 120(2), 120(4).

In some embodiments, a system may include the payload 100 and the inspection robot 10 described above, and may include a controller for control thereof.

In example embodiments including one or more sensing assemblies 120 positioned forward/offset from other sensing assemblies 120, as illustrated merely by example in FIGS. 15-16, such configurations may allow for improved horizontal resolution of an inspection operation, for example overcoming limitations or gaps that might be present in fully horizontally distributed arrangement and/or eliminating or reducing the need to raster the payload to achieve the desired inspection coverage.

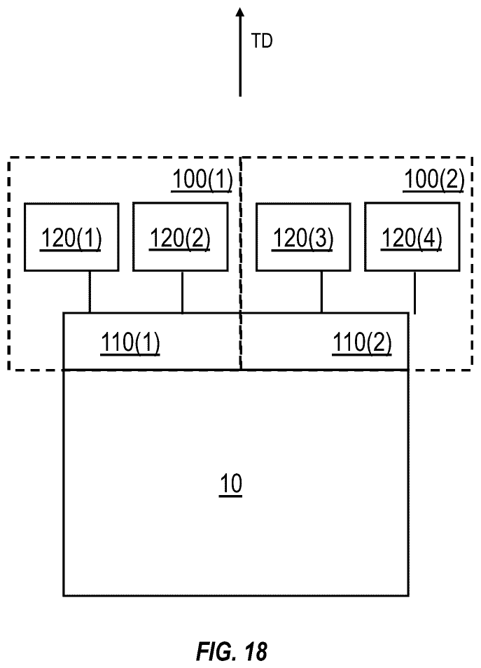

With reference to FIGS. 18-20, which illustrate schematic example configurations of an inspection robot 10 and payloads 100 with sensing assemblies 120, some example embodiments may include a plurality of payloads 100, each with one or more sensing assemblies 120. In some examples, the payloads 100 may each be rastered separately and/or together, and the sensing assemblies 120 within a given payload 100 may be the same or distinct. Additionally, the sensing assemblies 120 between the payloads 100 may be the same or distinct.

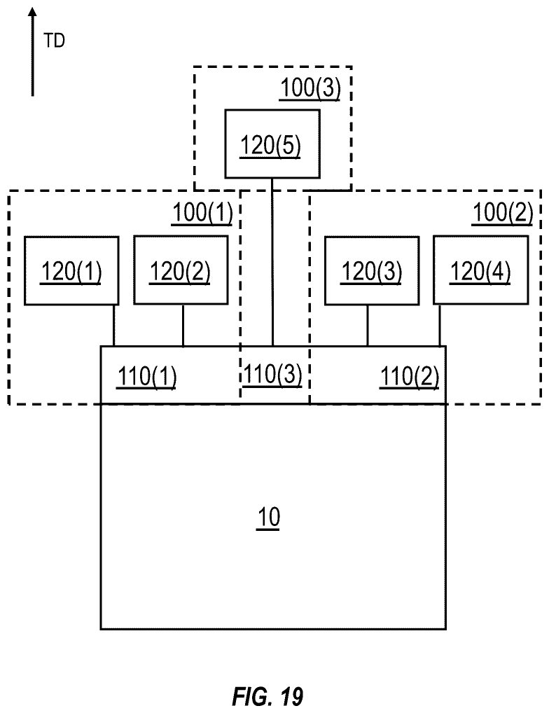

With reference to FIG. 19, in some example embodiments with a plurality of payloads 100, one or more forward payloads 100(3) may be positioned to cover some or all gaps between rearward payloads 100(1) and 100(2) and/or the sensing assemblies therein, and/or to provide an additional or distinct sensing package (e.g., combined with rastering, and/or for inspecting a feature expected to be at or near the centerline, such as a weld line).

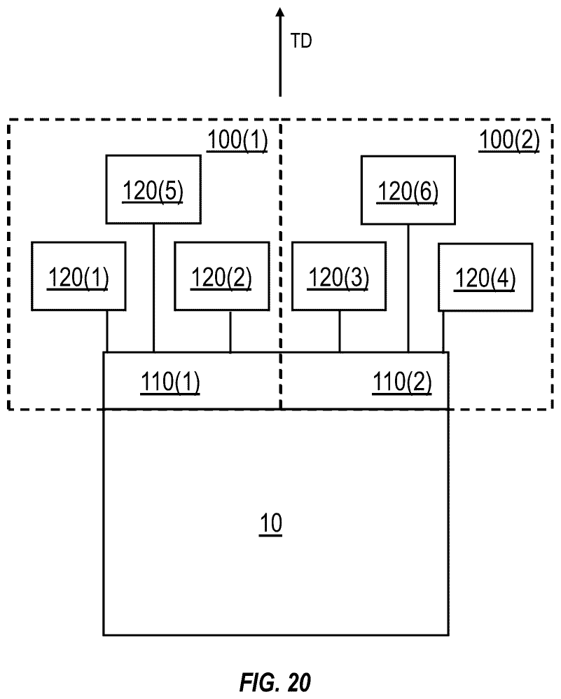

With reference to FIG. 20, in some example embodiments with a plurality of payloads 100, two or more horizontally distributed payloads 100 may be provided, where one or multiple ones of the payloads 100 includes vertically distributed sensing assemblies 120 to provide for arbitrary horizontal resolution of inspection operations and/or to inspect gaps between the rearward sensing assemblies. For example, payload 100(1) may include a sensing assembly 120(5) vertically forward of sensing assemblies 120(1) and 120(2), and payload 100(2) may include a sensing assembly 120(6) vertically forward of sensing assemblies 120(3) and 120(4).

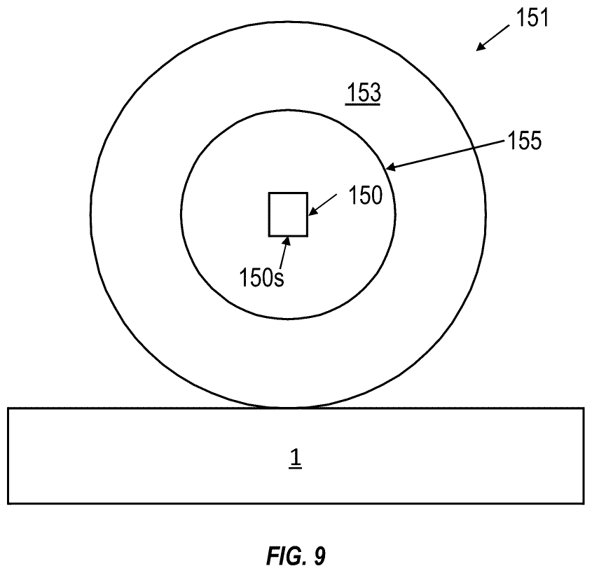

In example embodiments described herein, a payload 100 for an inspection robot 10 to inspect an inspection surface 1 may include a concrete sensing assembly 120 having at least a first transducer wheel 151 and at least a first impactor 140 structured to provide an acoustic impact against the inspection surface. With reference to FIGS. 9-12, which are schematic illustrations of example transducer wheels 151 on an inspection surface 1, the first transducer wheel 151 may include a first wheel 153 (also referred to herein as a wheel portion, as it forms a part of transducer wheel 151) and at least one transducer 150 to sense acoustic waves (which may, for example, include a compression wave and a shear wave) produced by the at least one impactor 140 providing the acoustic impact against the inspection surface 1. Consistent with example embodiments described herein, in some embodiments, the concrete sensing assembly 120 may include a plurality of impactors 140 and a plurality of transducer wheels including and/or each being a same or different embodiment of a transducer wheel 151 as described herein, and at least some of the transducer wheels 151 may be paired with and/or otherwise correspond with a respective one of the plurality of impactors 140.

The first transducer wheel 151 may include the at least one transducer 150 within the first wheel 153. Thus, the first wheel 153 (e.g., a material of the first wheel 153) may acoustically couple the at least one transducer 150 to the inspection surface 1. In some embodiments, the at least one transducer 150 may include a single transducer. For example, there may be no other acoustic transducers 150 included in the transducer wheel 151. Indeed, in certain embodiments, a single transducer 150 may be utilized, such as in a single transducer wheel 151 of the inspection assembly 120, with data collection and/or processing sequenced and planned to provide selected inspection operations.

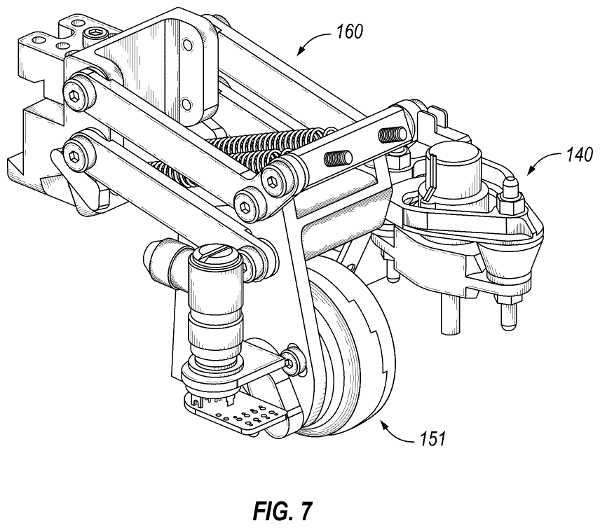

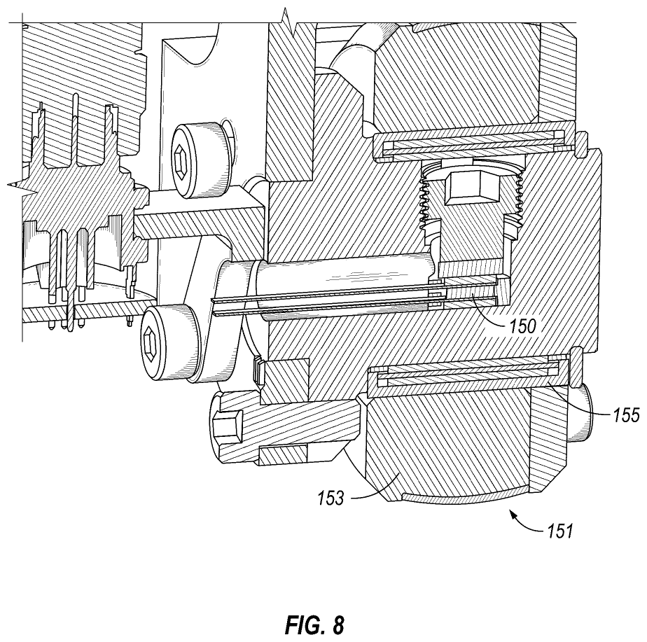

For example, FIG. 7 shows a first transducer wheel 151 paired with an impactor 140 in a transducer wheel assembly 160, including an impactor 140 and a transducer 150 (not shown) within a wheel shaft of a transducer wheel 151. In some embodiments, FIG. 7 may depict the entirety of a sensing assembly 120. FIG. 8 is an example cutaway view of an example sensing entity including a transducer 150 (e.g., a single transducer 150) within a wheel shaft of a transducer wheel 151 consistent with the example of FIG. 7. As shown in the schematic cross-section view of a transducer wheel 151 illustrated by FIG. 9, the transducer 150 may be within a bearing 155 of the transducer wheel 151. Additionally, the single transducer 150 may be fixed in position (e.g., on or within the wheel shaft within the transducer wheel 151) such that the single transducer 150 remains in a fixed orientation relative to the inspection surface 1 while the first wheel rotates on the inspection surface 1. For example, a sensing area 150s of the single transducer 150 may face downward in an orthogonal direction (which may be approximately orthogonal) to the inspection surface 1 to sense waves from the inspection surface 1 produced by the one or more impactors 140. A bolt may hold the transducer 150 in the fixed position.

The example embodiments illustrated in FIGS. 7-9 may utilize a single transducer 150 to perform sensing operations, for example in contrast to an arrangement with multiple transducers 150 of a transducer wheel 151 sequentially contacting and/or otherwise inspecting the inspection surface 1, which will be discussed below with reference to FIGS. 10-12. As discussed above, in the example of FIGS. 7 and 8, a wheel material of a wheel portion 153 of the transducer wheel 151 is utilized as at least part of the delay line, such that the wheel 151 acoustically couples the transducer 150 to the inspection surface. However, embodiments are not limited thereto, and in addition to and/or alternatively, in certain embodiments, the single transducer 150 may be coupled to the inspection surface utilizing a couplant chamber, for example with a radial through-hole that exposes the transducer 150 to a couplant filled chamber coupling the transducer 150 to the inspection surface through the through-hole.

Meanwhile, as described herein, the specific arrangement to acoustically couple the shaft mounted transducer 150 to the inspection surface is not limiting. In the example of FIG. 7, an optional impactor 140 is mounted to the side of the wheel. However, as described herein, one or more of the shaft mounted transducers 150 may not have an impactor 140. The utilization of a shaft mounted transducer provides for a number of benefits, including reducing the number of transducers 150 in the system for a given inspection capability, reducing the complexity of wheel couplings (e.g., in certain embodiments, the wheels do not have to have a fixed phase arrangement, and distances between wheels can be set variably), enables greater data density in both directions (e.g., vertical data density, for example generated by sampling frequency, can be arbitrarily selected as compared to sequential transducer contacts such as in FIGS. 10-12, and horizontal data density is simpler to enhance by adding wheels and therefore transducer contact locations), and wheels can be swapped to match acoustic properties of the inspection surface (e.g., allowing for an enhancement of inspection quality and/or a reduction in processing complexity).

Meanwhile, with reference to FIGS. 10-12, some example embodiments may include an arrangement with multiple transducers 150 of a transducer wheel 151 sequentially contacting and/or otherwise inspecting the inspection surface 1. FIGS. 10-12 illustrate example schematic cross-section views of such configurations. Indeed, in some embodiments, the at least one transducer 150 may include a plurality of transducers 150 facing radially outward from the first wheel 153, and may be outside of a bearing 155 of the transducer wheel 151. For example, a sensing area 150s of each transducer 150 may be orthogonal to the inspection surface 1 as the transducer 150 makes contact with or otherwise aligns with (e.g., through the wheel material and/or a coupling chamber) the inspection surface 1 to sense waves produced by the one or more impactors 140. By being located outside of the bearing 155, the bearing 155 may not interfere with acoustic wave sensing. A number of the plurality of transducers 150 for a transducer wheel 151 may be based on a desired inspection resolution, but in some examples, the number of the plurality of transducers 150 may be between three and twelve, inclusive. And in one example which may provide a balance of data demands (such as demands on analog-to-digital converters and/or subsequent processing), cost, and desired inspection resolution, the transducer wheel 151 may include six transducers 150.

Indeed, the number and spacing of the transducers 150 on a transducer wheel 151 may be selectable, for example according to the desired inspection resolution (e.g., the distance between inspected locations on the surface), available space for transducers, available electrical power capability to support a given number of transducers, and/or available data communication rates available to support inspection operations. The examples of FIGS. 2 and 10-12 include six transducers 150 positioned on each transducer wheel 151 as an example. However, embodiments are not limited thereto, and in certain embodiments, more or fewer transducers 150 may be utilized, and/or alternate transducer sequencing actuation may be utilized—for example raising and lowering transducers, as discussed herein. In certain embodiments, multiple transducers 150 may be maintained in contact with the inspection surface (e.g., with data collection and/or processing alternated in a selected sequence, and/or by transducers 150 included in different transducer wheels 151 and timed to be in contact with the inspection surface at a same time). In certain embodiments, the rolling transducer assembly, e.g., as provided by transducer wheels 151 including transducers 150 thereon/therein, may provide certain benefits, for example reducing physical wear of the transducer(s), providing a convenient tracking mechanism according utilizing the encoder 126 to confirm the present configuration of the concrete sensing assembly 120.

With reference to FIG. 10, as an example, the plurality of transducers 150 may be on an exterior of the first wheel 153 and may be spaced equally around a circumference of the first wheel 153. Thus, as the transducer wheel 151 rolls over the inspection surface 1, each of the plurality of transducers 150 may sequentially come into contact with and/or inspect the inspection surface 1. Indeed, the plurality of transducers 150 may be spaced around a circumference of the first wheel 153 such that each of the plurality of transducers 150 sequentially contacts the inspection surface as 1 the first wheel 153 rolls over the inspection surface 1. In example embodiments including a plurality of transducers 150 placed circumferentially around the transducer wheel 151, such as embodiments illustrated by example in FIGS. 10-12, the inspection robot 10 may time the one or more impactors 140 to impact the inspection surface 1 when a transducer 150 of the one or more transducer wheels 151 is positioned to inspect (e.g., is in contact with or orthogonally aligned with) the inspection surface 1. In an example, the timing determination may be made by a controller, such as a controller of the inspection robot 1, based on a reading from one or more encoders 126 indicating that a transducer 150 of the transducer wheel 151 is aligned for inspection. And as discussed above, owing to the relatively fast (e.g., 200-300 us) speed of inspection versus the relatively slow speed by which the transducer wheel 151 turns, such an inspection may be carried out while the inspection robot 10 is in motion or while the inspection robot 10 is stopped.

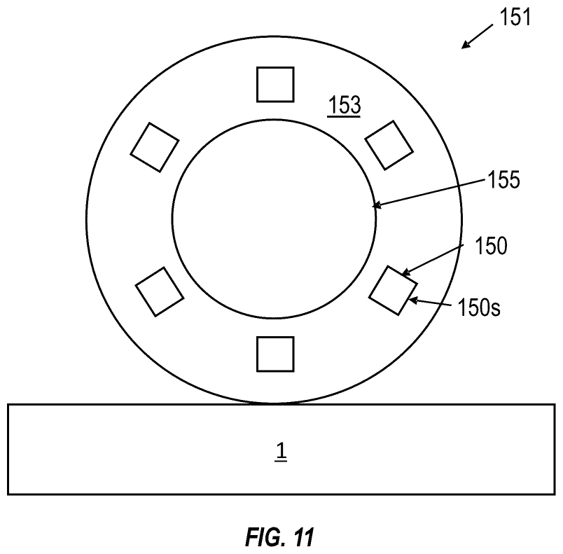

With reference to FIG. 11, in some embodiments, the plurality of transducers 150 are spaced apart from each other within a material of the first wheel 153. Additionally, as shown in FIG. 11, the material of the first wheel 153 may be between the transducers 150 and the inspection surface 1. Thus, as described by example herein, the material of the first wheel 153 may acoustically couple the transducers 150 to the inspection surface 1. And a material of the first wheel 153 may be selected to acoustically match or otherwise correspond (e.g., in a known acoustical relation) to the acoustic property/properties of the asset to be inspected, including the inspection surface 1. Indeed, in an example, an operator may select a transducer wheel 151 and/or sensing assembly 120 including the transducer wheel based on a material of the first wheel 153 used in the transducer wheel and a knowledge of its acoustic properties relative to the inspection surface 1. It should be noted that while example embodiments may refer to an inspection surface, such an inspection surface should be understood to refer to the surface of an asset to be inspected as well as the underlying material (e.g., cement or concrete) to be acoustically inspected via the impactors and transducers described in the example embodiments herein. An inspection surface may, in an example, comprise a metal surface and a concrete or cement layer on a side of the metal surface opposite to the inspection robot.

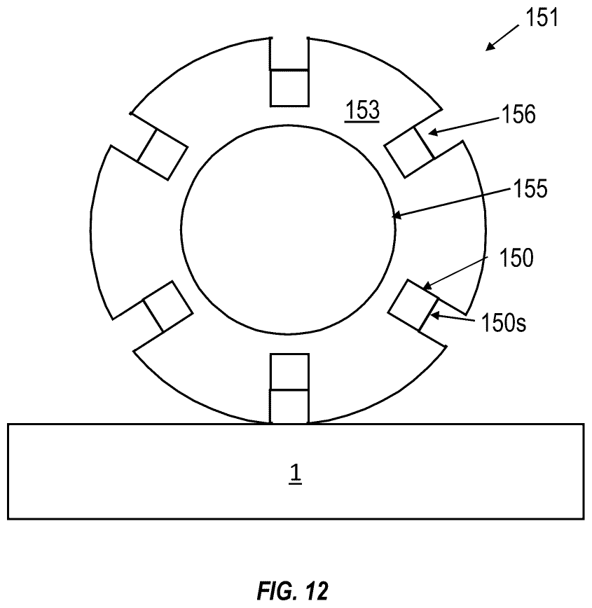

With reference to FIG. 12, and as noted above, in some embodiments, the plurality of transducers 150 are spaced apart from each other within a material of the first wheel 153. Meanwhile, in some embodiments, at least one transducer 150 (e.g., the plurality of transducers 150, although embodiments are not limited thereto) is radially inward of the inspection surface 1 when the wheel 153 contacts the inspection surface 1. In some embodiments, the at least one transducer 150 may be within a radial through-hole 156 of the wheel 153. For example, as illustrated in FIG. 12, the plurality of transducers 150 may be within radial through-holes 15 of the wheel 153. The radial through-holes 156 may provide couplant chambers to be filled with a couplant (e.g., water or another liquid) to acoustically couple each of the plurality of transducers 150 to the inspection surface 1 when, e.g., a radial through-hole 156 corresponding to a respective each of the plurality of transducers 150 is in contact with the inspection surface 1. In certain embodiments, the radial through-holes 156 may be filled with a material having a selected acoustic characteristic, for example a material with acoustic characteristics similar to those of the inspection surface, which allows for the wheel substrate material to be selected for mechanical properties or other characteristics.

Indeed, as described herein, the transducer 150 may be coupled to the inspection surface 1 utilizing a couplant chamber, for example with a number of radial through-holes 156 that sequentially expose the transducer 150 to a couplant filled chamber coupling the transducer 150 to the inspection surface 1 through the through-holes.

With reference to FIGS. 3 and 17B, in example embodiments, a payload 100 for an inspection robot 10 to inspect an inspection surface 1 may include a concrete sensing assembly 120 including a plurality of impactors 140 and a plurality of transducer wheels 151 distributed at respective horizontal positions along a horizontal axis HA. Each of the plurality of transducer wheels 151 may include a plurality of transducers 150 within a material of the transducer wheel 151. An orientation of the horizontal axis HA may be different from a direction of travel TD of the inspection robot 10. For example, the horizontal axis HA may be orthogonal (e.g., perpendicular) to the direction of travel TD of the inspection robot 10.

In example embodiments, there may be a greater number of the plurality of transducer wheels 151 than a number of the plurality of impactors 140. Additionally, the plurality of transducer wheels 151 and the plurality of impactors 140 may be distributed at the respective horizontal positions to provide for wave analysis at selected inspection depths, where such selected inspection depths are determined by distances between the plurality of transducer wheels 151 and the plurality of impactors 140 along the horizontal axis.

For example, FIG. 17B illustrates outermost transducer wheels 151(1) and 151(4) of the plurality of transducer wheels 151 at respective first and second ends of the concrete sensing assembly 120, where each corresponds to (e.g., may be paired with) respective outermost impactors 140(1) and 140(4) of the plurality of impactors 140 at the first and second ends of the concrete sensing assembly 120. For example, outermost impactor 140(1) may be paired with outermost transducer wheel 151(1), and outermost impactor 140(4) may be paired with outermost transducer wheel 151(4).

Indeed, in some embodiments, the plurality of impactors 140 may include only the outermost impactors 140(1) and 140(4) such that transducer wheels 151(2) and 151(3) of the plurality of transducer wheels 151 interior to the outermost transducer wheels 151(1), 151(4) do not correspond to respective impactors 140.

In some embodiments, as shown in FIGS. 17A and 17B, multiple ones of the plurality of transducer wheels 151 at different horizontal positions along the horizontal axis and at different distances (e.g., different horizontal distances) from one of the plurality of impactors 140 (e.g., each one of the plurality of impactors 140) may sense waves from the inspection surface produced by the one (e.g., each different one) of the plurality of impactors 140. The different distances may correlate to respective depths of inspection.

In some embodiments, with reference to FIG. 7, the payload 100 (e.g., the sensing assembly 120) may include a plurality of transducer wheel assemblies 160 each structured to maintain a fixed position between one of at least some of the plurality of transducer wheels 151 and a corresponding respective one of the plurality of impactors 140. Such a transducer wheel 151 maintained in a fixed position with an impactor 140 by a transducer wheel assembly 160 may be considered to be paired with or otherwise in correspondence with the impactor 140. Additionally and/or alternatively, a transducer wheel 151 may be considered to be paired with an impactor 140 by virtue of there being no coupling shaft 122 that enforces a spaced apart, designed distance between the transducer wheel 151 and impactor 140.

The utilization of transducers 150 as part of transducer wheels 151 (such as within a wheel shaft of the transducer wheel, on a circumference of the transducer wheel, or otherwise within the transducer wheel), combined with one or more impactors 140 in the sensing assembly 120, may allow for analysis between transducer-impactor pairs at varying distances and inspection depths, for example by performing surface wave analysis between pairs at varying distances. As described herein, in an example, each transducer may be paired with an impactor, allowing for a variety of surface wave analysis pairs to inspect varying depths of the inspection surface. In another example, the outer two transducers may be paired with an impactor, allowing for the same number of transducer-impactor distance pairs. The number of transducers, and thus the cost and complexity of the system, may be reduced, but the pitch of the compression wave analysis may be increased, and inspection using some transducer-impactor pairs at certain horizontal positions of the inspection surface may require some additional operations, such as increased rastering time.

The term payload, as used throughout the present disclosure, should be understood broadly. A payload, as used herein, includes any hardware element of the inspection robot that is capable of supporting at least two sensing entities, although in a specific configuration a payload may be supporting only a single sensing entity. Supporting includes any operations such as positioning (e.g., placing the sensing entity into operational contact with the inspection surface, raising or lowering the sensing entity, applying a downforce, rastering or otherwise applying horizontal mobility, etc.), mounting to the inspection robot, providing power, providing couplant, and/or providing communicative coupling (e.g., commands, data acquisition, status, etc.). The payload, in certain embodiments, is a term of convenience indicating an element of the inspection robot that is replaceable, in whole or part, allowing the payload to be swapped with another payload for any reason (e.g., to change the sensing package, to allow for maintenance or repair of a payload, to distribute wear between different payloads, to confirm proper operation of a payload, etc.). Accordingly, the physical elements of an inspection robot that make up a payload may vary between systems, and/or in a given system depending upon the specific configuration, reason for conceptualizing a hardware element as a payload, and/or upon the operating conditions of the inspection robot at a given time. For example, in certain embodiments, a sensing entity such as a concrete sensing assembly may be considered a payload. In certain embodiments, any subset (or all) of the components following may be considered a payload: a sensor mount or holder (e.g., a sled, mounting platform, etc.); an arm (e.g., allowing extension, movement, traversal of obstacles, enforcing geometric positioning, etc.) that couples to the sensing entity, sensor mount, or holder; and/or a mounting bracket (e.g., coupling the arm, sensor mount or holder, and/or sensing entity to the inspection robot). The terminology of a payload is not limiting to any particular embodiment. Any description of a payload herein relating to a particular set of hardware elements is a non-limiting example. One of skill in the art, having the benefit of the present disclosure and information ordinarily available about a particular system, can readily determine which hardware elements relate to a payload as utilized herein, and/or whether to consider a group of hardware elements as a payload. In certain embodiments, hardware elements may be configured as set forth in examples herein, without referencing any particular group of hardware elements as a “payload.”

A sensing entity, as used herein, includes any hardware elements such as a sensor, a group of elements configured to operate as a sensor (e.g., impactor-transducer assemblies), and/or related hardware elements to support the sensor—such as delay lines, couplant chambers, enforced spacing or phase angle components, or the like. The term sensing entity includes a sensor, but also includes aspects such as an assembly that operates as a sensor, where no single part of the assembly may ordinarily be identifiable as “the sensor.”

The methods and systems described herein may be deployed in part or in whole through a machine having a computer, computing device, processor, circuit, and/or server that executes computer readable instructions, program codes, instructions, and/or includes hardware configured to functionally execute one or more operations of the methods and systems herein. The terms computer, computing device, processor, circuit, and/or server, (“computing device”) as utilized herein, should be understood broadly.

An example computing device includes a computer of any type, capable to access instructions stored in communication thereto such as upon a non-transient computer readable medium, whereupon the computer performs operations of the computing device upon executing the instructions. In certain embodiments, such instructions themselves comprise a computing device. Additionally or alternatively, a computing device may be a separate hardware device, one or more computing resources distributed across hardware devices, and/or may include such aspects as logical circuits, embedded circuits, sensors, actuators, input and/or output devices, network and/or communication resources, memory resources of any type, processing resources of any type, and/or hardware devices configured to be responsive to determined conditions to functionally execute one or more operations of systems and methods herein.

Network and/or communication resources include, without limitation, local area network, wide area network, wireless, internet, or any other known communication resources and protocols. Example and non-limiting hardware and/or computing devices include, without limitation, a general-purpose computer, a server, an embedded computer, a mobile device, a virtual machine, and/or an emulated computing device. A computing device may be a distributed resource included as an aspect of several devices, included as an interoperable set of resources to perform described functions of the computing device, such that the distributed resources function together to perform the operations of the computing device. In certain embodiments, each computing device may be on separate hardware, and/or one or more hardware devices may include aspects of more than one computing device, for example as separately executable instructions stored on the device, and/or as logically partitioned aspects of a set of executable instructions, with some aspects comprising a part of one of a first computing device, and some aspects comprising a part of another of the computing devices.

A computing device may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more threads. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer readable instructions on a server, client, firewall, gateway, hub, router, or other such computer and/or networking hardware. The computer readable instructions may be associated with a server that may include a file server, print server, domain server, internet server, intranet server and other variants such as secondary server, host server, distributed server and the like. The server may include one or more of memories, processors, computer readable transitory and/or non-transitory media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, and the like. Additionally, this coupling and/or connection may facilitate remote execution of instructions across the network. The networking of some or all of these devices may facilitate parallel processing of program code, instructions, and/or programs at one or more locations without deviating from the scope of the disclosure. In addition, all the devices attached to the server through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods, program code, instructions, and/or programs may be associated with a client that may include a file client, print client, domain client, internet client, intranet client and other variants such as secondary client, host client, distributed client and the like. The client may include one or more of memories, processors, computer readable transitory and/or non-transitory media, storage media, ports (physical and virtual), communication devices, and interfaces capable of accessing other clients, servers, machines, and devices through a wired or a wireless medium, and the like. The methods, program code, instructions, and/or programs as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers, and the like. Additionally, this coupling and/or connection may facilitate remote execution of methods, program code, instructions, and/or programs across the network. The networking of some or all of these devices may facilitate parallel processing of methods, program code, instructions, and/or programs at one or more locations without deviating from the scope of the disclosure. In addition, all the devices attached to the client through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules, and/or components as known in the art. The computing and/or non-computing device(s) associated with the network infrastructure may include, apart from other components, a storage medium such as flash memory, buffer, stack, RAM, ROM and the like. The methods, program code, instructions, and/or programs described herein and elsewhere may be executed by one or more of the network infrastructural elements.

The methods, program code, instructions, and/or programs described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like.

The methods, program code, instructions, and/or programs described herein and elsewhere may be implemented on or through mobile devices. The mobile devices may include navigation devices, cell phones, mobile phones, mobile personal digital assistants, laptops, palmtops, netbooks, pagers, electronic books readers, music players and the like. These devices may include, apart from other components, a storage medium such as a flash memory, buffer, RAM, ROM and one or more computing devices. The computing devices associated with mobile devices may be enabled to execute methods, program code, instructions, and/or programs stored thereon. Alternatively, the mobile devices may be configured to execute instructions in collaboration with other devices. The mobile devices may communicate with base stations interfaced with servers and configured to execute methods, program code, instructions, and/or programs. The mobile devices may communicate on a peer-to-peer network, mesh network, or other communications network. The methods, program code, instructions, and/or programs may be stored on the storage medium associated with the server and executed by a computing device embedded within the server. The base station may include a computing device and a storage medium. The storage device may store methods, program code, instructions, and/or programs executed by the computing devices associated with the base station.

The methods, program code, instructions, and/or programs may be stored and/or accessed on machine readable transitory and/or non-transitory media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g. USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

Certain operations described herein include interpreting, receiving, and/or determining one or more values, parameters, inputs, data, or other information (“receiving data”). Operations to receive data include, without limitation: receiving data via a user input; receiving data over a network of any type; reading a data value from a memory location in communication with the receiving device; utilizing a default value as a received data value; estimating, calculating, or deriving a data value based on other information available to the receiving device; and/or updating any of these in response to a later received data value. In certain embodiments, a data value may be received by a first operation, and later updated by a second operation, as part of the receiving a data value. For example, when communications are down, intermittent, or interrupted, a first receiving operation may be performed, and when communications are restored an updated receiving operation may be performed.

Certain logical groupings of operations herein, for example methods or procedures of the current disclosure, are provided to illustrate aspects of the present disclosure. Operations described herein are schematically described and/or depicted, and operations may be combined, divided, re-ordered, added, or removed in a manner consistent with the disclosure herein. It is understood that the context of an operational description may require an ordering for one or more operations, and/or an order for one or more operations may be explicitly disclosed, but the order of operations should be understood broadly, where any equivalent grouping of operations to provide an equivalent outcome of operations is specifically contemplated herein. For example, if a value is used in one operational step, the determining of the value may be required before that operational step in certain contexts (e.g., where the time delay of data for an operation to achieve a certain effect is important), but may not be required before that operation step in other contexts (e.g. where usage of the value from a previous execution cycle of the operations would be sufficient for those purposes). Accordingly, in certain embodiments an order of operations and grouping of operations as described is explicitly contemplated herein, and in certain embodiments re-ordering, subdivision, and/or different grouping of operations is explicitly contemplated herein.

The methods and systems described herein may transform physical and/or or intangible items from one state to another. The methods and systems described herein may also transform data representing physical and/or intangible items from one state to another.

The methods and/or processes described above, and steps thereof, may be realized in hardware, program code, instructions, and/or programs or any combination of hardware and methods, program code, instructions, and/or programs suitable for a particular application. The hardware may include a dedicated computing device or specific computing device, a particular aspect or component of a specific computing device, and/or an arrangement of hardware components and/or logical circuits to perform one or more of the operations of a method and/or system. The processes may be realized in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and computer readable instructions, or any other machine capable of executing program instructions.

Thus, in one aspect, each method described above, and combinations thereof, may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or computer readable instructions described above. All such permutations and combinations are intended to fall within the scope of the present disclosure.

While the disclosure has been disclosed in connection with certain embodiments shown and described in detail, various modifications and improvements thereon will become readily apparent to those skilled in the art. Accordingly, the present disclosure is not to be limited by the specific examples described and depicted, but is to be understood in the broadest sense allowable by law.

1. A payload for an inspection robot to inspect an inspection surface, the payload comprising:
a concrete sensing assembly including a first impactor at a first end, a first near transducer at a first distance from the first impactor, and a second far transducer at a second distance from the first impactor, wherein the second distance is greater than the first distance; and
a payload mount structured to:
couple the concrete sensing assembly to the inspection robot; and
move the concrete sensing assembly between at least a first lowered position and a second raised position.

2. The payload of claim 1, further comprising:
the concrete sensing assembly further including a second impactor positioned at a second end, wherein the second end is opposite to the first end, the second far transducer is at the first distance from the second impactor, and the first near transducer is at the second distance from the second impactor.

3. The payload of claim 1, wherein:
when the concrete sensing assembly is in the first lowered position, the first impactor is in contact with the inspection surface; and
when the concrete sensing assembly is in the second raised position, the first impactor is spaced apart from the inspection surface.

4. The payload of claim 1, wherein the payload mount includes a downforce structure to provide a selected downforce of the concrete sensing assembly including the first impactor against the inspection surface.

5. (canceled)

6. The payload of claim 1, wherein the payload mount includes a spring-based structure having a plurality of springs to provide a selected downforce of the concrete sensing assembly against the inspection surface when the concrete sensing assembly is in the first lowered position and to provide a selected upforce of the concrete sensing assembly away from the inspection surface when the concrete sensing assembly is in the second raised position.

7. The payload of claim 1, wherein the payload mount includes at least one actuator structured to move the concrete sensing assembly between the first lowered position and the second raised position.

8. The payload of claim 1, wherein the first impactor includes a piezoelectric material to provide an impact against the inspection surface when the piezoelectric material is provided with an electric charge.

9. A system, comprising:
the payload of claim 1; and
the inspection robot.

10. The system of claim 9, wherein the inspection robot includes a controller to instruct the payload mount to move the concrete sensing assembly between the first lowered position and the second raised position.

11. The system of claim 10, wherein the controller instructs the payload mount to move the concrete sensing assembly to the second raised position when the inspection robot detects an obstacle on the inspection surface in a direction of travel of the inspection robot.

12. The system of claim 10, further comprising:
a first near transducer wheel including the first near transducer;
a second far transducer wheel including the second far transducer; and
another concrete sensing assembly including a third transducer wheel having a third transducer, wherein the controller instructs the payload mount to move the concrete sensing assembly between the first lowered position and the second raised position to change a sensing phase of at least one of the first near transducer or the second far transducer relative to the third transducer.

13.-19. (canceled)

20. A payload for an inspection robot to inspect an inspection surface, the payload comprising:
a plurality of concrete sensing assemblies including a first plurality of concrete sensing assemblies horizontally distributed across the payload relative to a direction of travel of the inspection robot,
wherein at least one of the first plurality of concrete sensing assemblies includes an impactor and a transducer; and
a payload mount structured to couple the concrete sensing assemblies to the inspection robot.

21. The payload of claim 20, wherein at least some of the plurality of concrete sensing assemblies include at least one transducer and at least one impactor.

22. The payload of claim 20, wherein the plurality of concrete sensing assemblies further includes at least one concrete sensing assembly positioned forward of the first plurality of concrete sensing assemblies relative to the direction of travel of the inspection robot to provide increased horizontal resolution of an inspection operation of the inspection surface.

23. The payload of claim 22, wherein the at least one concrete sensing assembly is positioned at a gap position to cover a horizontal gap between two or more of the first plurality of concrete sensing assemblies.

24. The payload of claim 23, wherein the at least one concrete sensing assembly is positioned at or near a horizontal center of the payload to inspect a feature of the inspection surface.

25. The payload of claim 24, wherein the feature includes a weld line.

26. The payload of claim 25, wherein the at least one concrete sensing assembly includes a sensing package that is distinct from a sensing package of at least one of the first plurality of concrete sensing assemblies.

27. The payload of claim 20, wherein the plurality of concrete sensing assemblies further includes a plurality of forward concrete sensing assemblies positioned forward of the first plurality of concrete assemblies relative to the direction of travel of the inspection robot, and wherein the plurality of forward concrete sensing assemblies are positioned at respective gap positions to cover horizontal gaps between respective two or more of the first plurality of concrete sensing assemblies.

28. A system, comprising:
the payload of claim 20; and
the inspection robot.

29.-55. (canceled)
